(19) United States
(12) Patent Application Publication
(10) Pub. No.: US 2022/0130527 A1
(43) Pub. Date: Apr. 28, 2022

(54) Treatment Sharing Method Implemented on a Treatment Machine

(71) Applicants: Fresenius Medical Care Deutschland GmbH, Bad Homburg (DE); Fresenius Medical Care AG & Co. KGaA, Bad Homburg (DE); Fresenius Medical Care Holdings, Inc., Waltham, MA (US)

(72) Inventors: Christina I. Allegrini, Hingham, MA (US); Harvey Cohen, Newton, MA (US); John Viero, Hong Kong (CN); Kirill Koulechov, Bad Vilbel (DE); Maria Millan-Galante, Bad Homburg (DE); Matthew Buraczenski, Holden, MA (US); Michael Thorwarth, Frankfurt am Main (DE); Paul von Buenau, Berlin (DE); Shashikant Dattatraya Kalaskar, Draper, UT (US); Stephen A. Merchant, Sudbury, MA (US); Thomas Stahl, Esselbach (DE); Thorsten Timm, Shanghai (CN); Nina Muellers, Bad Homburg (DE); Klaus Wolf, Muedesheim (DE); Erik Schumacher, Graefelfing (DE); Olaf Schermeier, Frankfurt am Main (DE); Zdenek Cerman, Idstein (DE); Christopher Hauke, Mainz-Kostheim (DE)

(21) Appl. No.: 17/429,421

(22) PCT Filed: Feb. 11, 2020
(86) PCT No.: PCT/EP2020/053371

§ 371 Date: Aug. 9, 2021

Related U.S. Application Data
(60) Provisional application No. 62/803,743, filed on Feb. 11, 2019.

(51) Int. Cl. G16H 40/20; G16H 40/63

(52) U.S. Cl. CPC G16H 40/20; G16H 40/63

(57) ABSTRACT

The present disclosure relates to a method for executing a sharing enabler of a registered medical treatment machine. The registered medical treatment machine is used in a shared medical treatment system. The method comprises the method steps:
Providing a registration procedure for registering the medical treatment machine in the shared medical treatment system;
Receiving an initiation request for initiating a medical treatment for a patient, being registered in the shared medical treatment system;
Checking whether the received initiation request is valid by receiving a confirmation signal and if yes:
Unlocking the medical treatment machine in reply to the received confirmation signal for use for the patient;
Operating the medical treatment machine according to a treatment procedure in a sharing mode.

### CROSS-REFERENCE TO RELATED APPLICATIONS

The present application is the national stage entry of International Patent Application No. PCT/EP2020/053371, filed on Feb. 11, 2020, and claims priority to U.S. Provisional Application No. 62/803,743, filed on Feb. 11, 2019, the disclosures of which are expressly incorporated herein in their entireties by reference thereto.

### TECHNICAL FIELD

The present disclosure generally relates to the field of medical treatment by machines, e.g., dialysis machines or other blood treatment machines, and to an approach for sharing dialysis treatment or other medical treatment services using treatment sharing methods implemented on registered medical treatment machines.

### BACKGROUND

Medical treatment machines can be designed to aid in the diagnosis, monitoring, and/or treatment of a variety of medical conditions. One example of a medical treatment machine is a dialysis machine. Dialysis is a treatment used to support a patient with insufficient renal function. The two principal dialysis methods are hemodialysis and peritoneal dialysis.

During hemodialysis (“HD”), the patient's blood is passed through a dialyzer of the dialysis machine while also passing a dialysis solution or dialysate through the dialyzer. Thus, the patient's blood is temporarily brought outside of the body with the help of tubes and passed through at least one semipermeable membrane, which may be a group of hollow fibers, in an artificial kidney, also called a dialyzer. The semi-permeable membrane in the dialyzer separates the blood from the dialysate within the dialyzer and allows diffusion and osmosis exchanges to take place between the dialysate and the blood stream. These exchanges across the membrane result in the removal of waste products, including solutes like urea and creatinine, from the blood. These exchanges also regulate the levels of other substances, such as sodium and water in the blood. In this way, the dialysis machine acts as an artificial kidney for cleaning the blood. During this procedure it is also necessary to remove excess fluids from the body. This is accomplished by a process known as ultrafiltration. In this process, fluid is removed from the patient by taking the fluid off through the dialyzer via convection and discarding it. The amount of ultrafiltrate which is removed from the body is normally controlled by the pressure across the semipermeable membrane. This transmembrane pressure is the result of the differential between the blood pressure and the pressure which exists on the dialysate side of the membrane. Accordingly, the treatment may comprise two phases, namely, (1) true dialysis, in which toxic substances and scoriae (normally small molecules) pass through the semipermeable membrane from the blood to the dialysis liquid, and (2) ultrafiltration, in which a pressure difference between the blood circuit and the circuit for the dialysis liquid, e.g., a reduced pressure in the latter circuit, causes the blood content of water to be reduced by a predetermined amount. In an alternate procedure to hemodialysis, known as hemofiltration, convection is used to withdraw massive amounts of fluid from the body via the dialyzer and most of that volume is replaced by ultrapure, infusate grade, fluid pumped directly into the blood stream. In this process the ultrafiltrate removal volume is the difference between the amount of fluid removed and the amount of ultrapure infusate injected. Hemofiltration is better at removing large molecular toxins than hemodialysis but is not required in most cases.

Hemodiafiltration combines the methods of hemodialysis and hemofiltration. A diffuse mass exchange takes place between the patient's blood and the dialysis fluid through the semipermeable membrane of a dialysis machine as well as filtering of plasma water through a pressure gradient on the membrane of the dialysis machine.

During peritoneal dialysis (“PD”), the patient's peritoneal cavity is periodically infused with dialysate. The membranous lining of the patient's peritoneum acts as a natural semi-permeable membrane that allows diffusion and osmosis exchanges to take place between the solution and the blood stream. These exchanges across the patient's peritoneum result in the removal of waste products, including solutes like urea and creatinine, from the blood, and regulate the levels of other substances, such as sodium and water, in the blood.

Automated PD machines called PD cyclers are designed to control the entire PD process so that it can be performed at home usually overnight without clinical staff in attendance. This process is termed continuous cycler-assisted PD (“CCPD”). Many PD cyclers are designed to automatically infuse, dwell, and drain dialysate to and from the patient's peritoneal cavity. The treatment typically lasts for several hours, often beginning with an initial drain cycle to empty the peritoneal cavity of used or spent dialysate. The sequence then proceeds through the succession of fill, dwell, and drain phases that follow one after the other. Each phase is called a cycle.

Irrespective of the particular treatment (mentioned above) to be applied to the patient, conventional dialysis procedures using standard medical treatment services and equipment tend to be cumbersome as well as costly, besides requiring the patient to be bound to a dialysis center or to use a “private” machine at home, which will only be used during the patient's treatment and will remain unused in the rest of the time. Some types of the conventional systems are also less flexible because of the necessity of using a treatment facility with medical services at a specific time and location. Especially, in rural areas, patients need to take a long trip to get to the next available treatment facility. This requires extensive and foresighted planning for each medical treatment. Further, some treatment machines may be purchased on a private basis and may be operated on behalf of a special patient. As a consequence, this machine is only active during a short time period and is inactive for the rest of the time. Taking into account the duration of application for one treatment, the machine therefore remains unused for most of the time. This may lead to a period of time in which the treatment machine is unused, which could however be used for other treatments of other patients and thus, represents a loss of valuable treatment time. Further, inactive treatment machines may lead to increasing costs in maintenance due to the fact that more treatment machines are required for treatment of patients then in a sharing scenario. Further, in the state of the art, medical treatment machines and/or center having a plurality of medical treatment machines performing dialysis treatments independently of each other, without appropriate coordination and possible loads and capacity distribution. This is due to the lack of communication connections between each other. This may lead to a loss of treatment time as well as multiple provision of capacities, e.g. consumables, supervision etc.

Accordingly, there is a need in the art for an approach that supports machines of providers participating in a sharing system for sharing blood treatment services and machines that can provide dialysis treatment, while at the same offering a self-treatment option for the patient at the provider in a controlled and supervised manner. Especially, there is a need in the art for an approach, where treatment machines actively participate and communicate to each other, to a patient, emergency provider, and/or physician for exchanging data and offering their services when they are currently available. Further, there is also a need for a novel approach for a treatment sharing method that enables a monitored treatment service provided by the treatment machine alone in an unmanned facility or a facility without medical service being provided locally. Moreover, problems should be avoided (e.g., for the patient) in case the provider or the machine is or becomes not eligible for offering a treatment option—e.g., a self-treatment option—anymore.

Accordingly, it is advantageous to provide an approach that enables a sharing of dialysis treatment services by the dialysis treatment machines.

Advantageous modifications, refinements and options are described in the description with reference to the drawings.

According to a first aspect, this advantage is provided by a method for executing a sharing enabler of a registered medical treatment machine, to be used in a shared medical treatment system. The method can be implemented on an electronic module of a medical treatment machine. The method comprises the steps:
Providing a registration procedure for registering the medical treatment machine in the shared medical treatment system;
Receiving an initiation request for initiating a medical treatment for a patient, being registered in the shared medical treatment system;
Checking whether the received initiation request is valid by receiving a confirmation signal and if yes:
Unlocking the medical treatment machine in reply to the received confirmation signal for use for the patient;
Operating the medical treatment machine according to a treatment procedure in a sharing mode.

In the following the terms used within this application are defined in more detail.

A patient device is an electronic device that a patient may use and that is configured to plan, organize, prepare, and/or refinish the medical treatment of a patient. The electronic device may comprise certain interfaces for handling the patient device and communication with the patient and/or further electronic devices, such as computing devices, e.g., server, treatment machines, and further communication devices used by people responsible for the treatment of the patient, e.g., a physician.

The provider is a provider for medical treatment services, e.g., dialyses services. The provider may be a private person, offering his or her private dialysis machine to other registered patients. The provider may be an institution for offering medical treatment services, like a medical center, a hospital, a clinic, a clinical department, or an operator of an unserved and/or unmanned dialysis machine park. The provider provides a set of medical treatment machines, e.g., dialysis machines. The provider administers and controls and operates the set of machines. The set of machines may comprise one single machine (in the case of the provider being a private patient offering his or her machine) or may comprise a set of machines (e.g., the provider being a dialysis center). Thus, the provider serves as host for medical services. The provider has, comprises, and/or provides treatment machines. The provider is registered in a hub and is in data exchange with this hub. A registered provider with medical treatment machines is a provider that is enrolled for use and allowed to participate in the shared medical treatment system for providing treatment services and/or treatment machines.

A provider device is an electronic device that a provider may use and that is configured to plan, organize, prepare, and/or refinish the medical treatment of a patient. The electronic device may comprise certain interfaces for handling the provider device and communication with the patient and/or further electronic devices, such as computing devices, e.g., server, treatment machines, and further communication devices used by people responsible for the treatment of the patient, e.g., a physician, nurse, and/or emergency provider. Further, the provider device can be used to setup up and/or program the treatment machines as well as the treatment.

A physician device is an electronic device that a physician may use and that is configured to prepare the medical treatment of the patient. Preparing the medical treatment of the patient may comprise issuing a prescription comprising requirements or the specification of the medical treatment. The electronic device may comprise certain interfaces for handling the physician device and for communication with the patient and/or further electronic devices, such as computing devices, e.g., server, treatment machines, and further communication devices used by people responsible for the treatment of the patient, e.g., a nurse and/or emergency provider. Further, a physician may use the physician device for supervising the medical treatment, providing support in case of malfunction or emergency, and/or confirming eligibility of a patient for performing self-treatment.

In the present context, said electronic device can be designed as a handheld. A handheld can be a portable electronic device that is powered by rechargeable batteries or once used batteries and can be used for various applications. A handheld provides the advantages that it is small and light, which enables to hold and control it only in one hand during operation. A handheld in the present context can be, for instance, a personal digital assistant (PDA), a tablet computer (tablet), a smartphone, a portable data terminal (PDT), etc. In an embodiment, said electronic device can be designed as a watch, e.g., as a smartwatch that is wearable at the wrist.

An electronic device may comprise electronic modules that may form an interrelated computing entity. The computing entity may comprise a plurality of different electronic modules with corresponding features selected according to the application of the electronic device. The electronic modules of the electronic device are connected to each other by a communication bus. An electronic module implemented in the electronic device can be, for instance, a central processing unit (CPU) for processing software, e.g., mobile applications (APP), a volatile and non-volatile memory for storing data, e.g., an operation system, mobile applications, databases, user data. The memory can be implemented separately or in the CPU. Further, the electronic device may comprise a user interface, e.g., a graphical user interface in combination with a touchscreen configured to, both, input and output data. The touchscreen can be layered on the top of the electronic device to enable controlling of the electronic device by a user, e.g., the patient. Further, the electronic module may comprise interfaces for connecting the electronic device to a network, e.g., local network (LAN), the internet (WAN), for communicating with further computing entities such as computer, server, treatment machines, or other communication devices. The electronic device may comprise further interfaces, which support communication protocols such as WI-FI, Bluetooth, Bluetooth low energy, Infrared, UMTS, LTE, and 5G. This has the advantage that the electronic device can be connected to other electronic devices and networks wirelessly for communication, upgrade and update of software, and/or data exchange. In a further embodiment, the electronic module may comprise a serial interface, e.g., an interface using an USB standard for establishing a wired connection. This has the advantage that the electronic device can be connected to other electronic devices for communication, upgrade and update of software, and data exchange. Using the USB4 standard, the interface can be used to charge the rechargeable batteries of the electronic device. In a further embodiment, the electronic device may comprise various sensors for measuring vital parameter, e.g., weight, blood pressure, a body composition index (which may be provided on a body composition monitor: BCM), oxygen saturation, blood sugar and/or general impression of the patient, detecting and measuring the position and the environment around the position of the user carrying the electronic device. In an embodiment, the sensor can be implemented in an additional device, e.g., a chest strap for measuring the vital parameter. The chest strap can be connected to the electronic device by a wireless communication connection. The electronic device may comprise further interfaces, such as a camera, speaker for communication and/or for sending and receiving audio and/or visual data.

According to the present disclosure, a sharing enabler can be a software routine that can be processed by a computing entity comprising a processing unit. The computing entity can be integrated in the treatment machine. The sharing enabler may comprise instructions to perform the method according to the present disclosure. Further, the sharing enabler may be implemented as an electronic module, e.g., a microcontroller as a part of the computing entity. The sharing enabler can be changed by upgrading and/or updating, for instance, for bug-fixing, for implementing new features and/or treatment capabilities.

The medical treatment or related services may be a blood treatment, e.g., a dialysis treatment and more particular a self-treatment, which may be supervised or unsupervised. Related services may refer to services related to medical treatment, e.g., to reservation services, machine setup services, scheduling services, and others. The type of treatment may be defined in a prescription message. In a self-treatment, the patient may procure its own consumables and may carries out the treatment itself according to predetermined steps. In this respect, the patient may attend successfully certain training courses beforehand and acquire or prove certificates that he/she is able to perform this treatment. The medical treatment may provide an online service for support and/or technical service and/or emergency support by using various communication channels. In a supervised treatment, the medical treatment of the patient can be performed by a nurse, physician, and/or an appropriately trained third person.

In the present context, a shared medical treatment system provides an approach for sharing medical treatment. The shared medical treatment system provides a network for communication of different parties involved in the medical treatment process such as, the patient, physician or nurse, provider of treatment service, and the treatment machines. The communication is administrated by a central computing entity or services of a computing entity distributed to a plurality of computing units. The shared medical treatment system can be designed as a medico-computer system for providing a sharing platform for sharing medical treatment sharing services. The respective medical machines (e.g., dialysis machines) and/or the computers (e.g., app or hub server) may be located at different positions and are therefore locally distributed and in data connection with each other (e.g., at least portions thereof are implemented as wireless data connection, the data connection is implemented on a secured data communication protocol). The shared medical treatment system comprises a plurality of machines, a plurality of patients, each patient using his/her own patient app, a computing entity and/or a hub. The computing entity may be provided in the hub.

The hub is a computer entity. The hub may be implemented as central server, computer, or network. The hub may even be hosted on a server or may be virtualized. The hub may also be distributed over a set of computing entities. The hub can be a computer-based administration unit, and may be implemented as a central connection point. The hub may be connected to a set of providers with its treatment machines, to a set of patient devices (with patient apps), to a set of physician devices (with physicians' apps), to a set of emergency providers, to a database, and/or to a consumables administration unit etc. The computing entity may be a computer and/or an embedded device. The computing entity may also be a processor or a processing unit.

A medical treatment machine in the present context is preferably a blood treatment machine performing a hemodialysis and/or peritoneal dialysis treatment. A registered medical treatment machine is a treatment machine that is enrolled for use and allowed to participate in the shared medical treatment system for providing medical treatment. A treatment machine is registered after a successful participation in a registration process. The registration process is configurable and may comprise a plurality of registration functions, like providing information for kind of machine and services that can be provided, location information, available time slots for performing a medical treatment, provided consumables, proofing/fulfilling payment requirements, and/or the like. The registration procedure can be used to exchange all relevant and required data between the treatment machine, the provider of the treatment machine, and the hub, patient, and/or physician.

Further, the treatment machine may be operated in a locked and an unlocked mode. In the unlocked mode all functionalities of the treatment machine for performing a medical treatment are available and can be retrieved by a patient. For unlocking a treatment machine, a confirmation signal is required. In the locked mode all functionalities of the treatment machine are disabled and cannot be retrieved by a patient. The mode can be switched, respectively, the treatment machine can be activated/deactivated by the provider of the treatment machine. This functionality increases safety against unauthorized or unconscious incorrect use of the treatment machine.

A registered patient is a user that is enrolled for use and allowed to participate in the shared medical treatment system for requesting medical treatment. A user is registered after successful participation in a registration process. The registration process is configurable and may comprise a plurality of registration functions, like filling a questionnaire on eligibility for self-treatment, determination of a stable vascular access, finishing trainings, and/or proofing/fulfilling payment requirements. The determination of a stable and correct vascular access is essential for patient's safety. Usually, an arteriovenous fistula is created for access to the vascular system, but an implant may also be used. Blood is taken from the patient through an arterial needle connected to the arterial blood line of the extracorporeal blood circulation loop, and then it is supplied back to the patient again through a venous needle connected to the venous blood line. The kind and the condition of the vascular access may be determined.

A selected registered patient is a registered patient who has been selected by a matching algorithm to be associated to a medical treatment machine. A selected registered patient may receive the matching result with a set of medical treatment machines. The set of suggested treatment machines is matching the selected patient's requirements.

A registered patient may have a patient identifier. The patient identifiers serve to uniquely identify a particular patient. The patient identifier may be an electronic code, e.g., in form of an electronic fingerprint, a data structure or a biometrical dataset. Generally, a patient needs to register in the shared medical treatment system. Thus, not all patients are registered patients. The patient identifier can be combined with an additional secret, e.g., a password comprising a random combination of numbers, letters, and/or characters. The password has to fulfill specific criteria relating to length and combination.

An initiation request may comprise an electronical signal or request from a patient device of a patient, a computing entity, e.g., a hub, server, for initiating a medical treatment. The initiation request may comprise the start for technical and administrative preparation of the treatment machine according to the requirements for the medical treatment requested by a specific patient. The initiation request has to be confirmed by a confirmation signal. If no confirmation signal is received, the initiation of the treatment machine is paused and a reminder in form of a message can be send to the requesting patient and/or to the confirming party. If the reminder is not taken into account, the initiation is stopped and the medical treatment is not performed. In this case, the patient can be referred to another provider and/or hospital for performing his/her medical treatment to avoid possible medical complications.

A confirmation signal is an electronic signal that is generated by a central processing unit of an electronic device and is transmitted/received to/by another electronic device using a communication interface of the electronic device. The confirmation signal may comprise a bit structure signalizing the confirmation as yes respectively “1” and as no respectively “0”, and/or reverse. In an embodiment, the confirmation signal may comprise a data structure including the confirmation and/or further information. The confirmation signal may be generated by the physician device, respectively the physician or provider device respectively the treatment provider, or other devices but in any case, a device other than the patient's device. The physician may confirm with the confirmation signal the correctness of the transmitted prescription and/or the requested medical treatment and the required and selected consumable(s). The provider may confirm with the confirmation signal the availability of the required consumable to carry out the medical treatment. Further the confirmation signal may be further issued by the treatment machine or by a hub. This has the advantage that a treatment service provider confirms as well as accepts the request sent by the patient application. The patient application is executed on the patient device. In an alternative embodiment, it is also possible that a confirmation is expected to be provided by the user of the patient application (i.e., the patient). This has the advantage that the patient may finally accept or refuse the automatically calculated suggestion in the message.

The methods according to this disclosure have several advantages. The present disclosure enables a faster and more efficient planning, preparation, and executing of a medical treatment. The methods enable a quicker deployment of required treatment machines and performing of the medical treatment. This is achieved on the one hand by faster registration, as well as by automatic verification of the patient to be treated and their prescription. In result, it enables a quicker and more efficient assignment of treatment time.

Further, by using the methods according to the present disclosure, the treatment machines may be faster and more efficient equipped with the required consumables for each individual medical treatment. A medical treatment, e.g., a dialysis treatment is not as any other. Moreover, the treatment machine may be setup up with different hardware and has to be configured differently. Requirements for the treatment, as well as settings in the treatment machine, which are known in advance, can be efficiently planned and controlled, and/or the patient can be informed at an early stage that a medical treatment as requested cannot be carried out at present.

Advantageously, the methods provide an improved and more efficient exchange of treatment information between the patient, the provider of treatment machines and/or the treatment machines, as well as the physician. Therefore, it is possible to act faster in case of any treatment problem or disturbance.

Moreover, the present methods may improve the security for dialysis treatment, e.g., self-treatment/medical treatment on a network of providers through performing a supervision by using sensors equipped in the treatment machine or in the providers location.

Moreover, the present methods may prevent problems of a patient and/or incorrect operation, e.g., in case of self-treatment of a patient, who is not eligible for self-treatment.

In some advantageous embodiments, the medical treatment machine is hosted by a provider, who may be a patient himself. This can be the case, if a provider who needs the treatment machine for treatment himself makes the treatment machine available in the non-usage phase. In time periods when the treatment machine is not in use, the treatment machine may register to the shared medical treatment system and provides the unused time-periods for other patients seeking time periods for their medical treatment. In this way, the provider or the treatment machine may provide all required data (e.g. free time periods, location, address of location, type of treatment machine and feasible treatments, required consumable (provided or has to owned by the patient), certification, etc.) to perform a medical treatment. These data can be used to rank, verify, rate, and/or submit the provider to potential patients. Therefore, the unused time-periods can be reduced and costs for the machine can be shared. Further, a group of people living in a near neighborhood may purchase together a treatment machine and can use the method for organizing the sharing of the treatment machine as well as the administration of the stock for consumables.

In an alternative embodiment, the provider can be a clinic. A clinic can be specialized in providing dialysis treatment. The clinic may provide a variety of treatment machines with different consumables. The clinic may provide supervised treatment or self-treatment. Further, the clinic may provide assistance and/or emergency provider on cite. The method may provide automatically register the treatment machines and provides corresponding services to the patients. In an embodiment, the clinic may provide 24/7 support for dialysis self-treatment. A patient may have an access Id-card that allows to enter the clinic. The required consumable can be provided according to the request, or the patient brings the consumables him/herself and equip the treatment machine. The setup of the program for the medical treatment has been performed automatically according to the in advanced provided prescription.

In a further alternative embodiment, the provider may be a medical or non-medical institution. In this way, a medical institution, e.g., a physician with a different specialization may provide in his/her office space for performing a dialysis treatment. In this way, the physician may provide a treatment machine, or the patient may bring his/her own treatment machine. The physician may provide in emergency situation first aid. In a non-medical institution, only treatment machines or space for bringing and using the own treatment machine is provided.

In some advantageous embodiments, checking validity (of the received initiation request) comprises checking availability of consumables necessary for executing the requested medical treatment. Checking availability of consumables, e.g., of required consumables, is performed while performing a validation of the received initiation request. It is necessary to check available consumables at the early stage and before starting preparation of the treatment machine for the medical treatment. A medical treatment may need specific, and in each case different consumables. The prescription for the medical treatment may define that a patient needs to use a specific type of consumables or disposables, like tube sets, filters, adapters etc. Thus, it has to be assured that this kind of type of consumables is available at the treatment machine and, e.g., available at the scheduled treatment time. For instance, the method may be configured to check storage of consumables at the specific treatment machine or at the location of the treatment machine, and to replenish stock of consumables, e.g., after each treatment.

In some advantageous embodiments, checking validity comprises checking availability of necessary emergency providers for the requested medical treatment. Checking availability of emergency providers is performed while performing a validation of the received initiation request. A dialysis treatment is an intervention in the human circulatory system where complications can occur that can endanger health and/or life of the patient. In addition, complications can occur at any time, both in terms of technical complications with the machine and also with the patient's tolerance of the treatment. Therefore, a medical treatment can only be performed, respectively, the treatment machine can be unlocked, if the availability of necessary emergency providers for the requested medical treatment is confirmed. In this way, the treatment machine is only unlocked, if an emergency provider, e.g., a physician or nurse, or a third person trained for the medical treatment, for the requested medical treatment is available. The emergency provider may support the patient in using the treatment machine in case of technical issues or supports in case of physical emergency, e.g., first aid of the patient. The availability may be ensured by personal presence of a physician, nurse or other third parties, by online availability (audio and video conference), or by fast on-site availability.

In some advantageous embodiments, operating the medical treatment machine is based on a prescription, received from a physician's device via a network connection. The treatment machine may comprise an interface that is configured to establish a wireless or a wired communication connection to a communication network. The network connection may be used for connecting the treatment machine to a hub and/or a patient device and/or a physician device and/or any communication device of an emergency provider. The interface may be an ethernet interface for connecting the medical treatment machine to a LAN, WAN, MAN network. In a further embodiment, the interface may be configured to support communication protocols such as Wi-Fi, Bluetooth, Bluetooth low energy, Infrared, UMTS, LTE, and 5G. This has the advantage that the treatment machine can be connected to other electronic devices and networks wireless for communication, upgrade and update of software (e.g., share enabler), and data exchange.

In some advantageous embodiments, the registration procedure for registering the medical treatment machine comprises checking a set of certificates. Different certificates can be raised for different capabilities. The set of certificates therefore may include pre-configurable provider certificates and/or preconfigurable patient certificates. The certification is processed by a certification function. A certification function is a procedure for certifying that the machine is designed to perform specific medical treatment or that the location of the treatment machine and/or the treatment machine itself fulfil expected requirements. Further, the certification function may certify that a patient has or has acquired knowledge and capabilities with respect to required procedures or activities, related with a self-treatment procedure. In this context, the preconfigurable provider certificate may comprise an eligibility certificate, a machine certificate, an infrastructure certificate, a hygiene certificate, an emergency certificate, a usage certificate, and/or a maintenance certificate. The proof of the certificates as well as their verification may improve the security and quality of the shared medical treatment system by assuring that all necessary and actual requirements for becoming a provider or service host are met. The requirements may change over time. The requirements may differ from provider to provider.

Further, the preconfigurable patient certificate may comprise a self-treatment ability certificate and/or a set of training certificates. The proof of the certificates as well as their verification may improve the security and quality of the shared medical treatment system by assuring that all necessary and actual requirements for becoming a self-treating patient and thus for participating in the shared medical treatment system as registered patient are met. The requirements may change over time. The requirements may differ from patient to patient (e.g., based on the physician's prescription).

Further, the set of certificates may relate to fault and/or alarm handling, to specific machine certificates, and/or specific consumables certificates.

In some advantageous embodiments, the method according to the first aspect may comprise establishing a network connection to a hub system. In some advantageous embodiments, the method according to the first aspect may comprise establishing a network connection to a treatment provider's device. In some advantageous embodiments, the method according to the first aspect may comprise establishing a network connection to a physician's device. The treatment machine may establish a network connection to different further systems and/or devices by using its certain interfaces. A network connection to the hub system may enable the data communication and data exchange between the treatment machine and the hub system as computer entity and central connection point of the shared medical treatment system. A network connection to the treatment provider device may enable the data communication and data exchange between the treatment machine and the treatment provider. The treatment provider may use the provider device to confirm the patient request, manually unlock the treatment machine, start the medical treatment, and/or for maintenance purposes. A network connection to the physician's device may enable the data communication and data exchange between the treatment machine and the physician and/or a nurse. The physician may verify the certification, supervise the medical treatment by receiving current treatment machine data and/or current vital parameters of the patient.

In some advantageous embodiments, the communication connection between an electronic device (provider device, physician device, patient device, etc.) and a further communication device (hub system, treatment machine, etc.) is secured by using a secure message transfer. The secure message transfer comprises in an embodiment secure HTTP (S-HTTP). S-HTTP is an extension of the existing HTTP protocol and is based on three types of security: signature, authentication and encryption. S-HTTP encapsulates ordinary HTTP messages. The encapsulated requests and responses are either encrypted or signed, encrypted and signed or neither. This increases the security of the communication connection. In a further embodiment, the secure message transfer comprises secure socket layer (SSL). SSL places an additional layer above the socket layer. This layer looks like the usual socket layer, i.e., towards the application layer. Compared to the actual sockets, it appears as an application. This security protocol enables data encryption, authentication of servers and ensuring message integrity for TCP/IP connections. Secure Socket Layer always provides the client with server authentication. Therefore, the server operator always requires a certificate from a certification authority. The authentication of the client side is optional and is only successful if the client has an officially registered certificate. Once the SSL connection between a client and a server has been established, all data is encrypted and thus transmitted unreadable to a third party. All information, both the HTTP request and the HTTP response, is fully encrypted. This includes the URL requested by the client, the information transmitted in forms, the information for confirming the authenticity of HTTP access, and all data that the server transmits to the client. SSL uses the public-key method, in which data encoded with a publicly accessible key can only be decrypted again with a very specific private key. An integrity check in SSL detects any falsification of the data sent.

In some advantageous embodiments, the method and checking validity comprises sending a provider confirmation request to a treatment provider. In this context, checking the validity may comprise checking the received initiation request. In this way, the provider selected and requested for a medical treatment by a patient confirms that he/she has the capacities, treatment machines, consumables in a specific time period available. A first part of a handshake is performed to ensure the medical treatment.

In some advantageous embodiments, the method and checking validity comprises receiving a confirmation signal of the treatment provider, indicating that the treatment provider confirms itself for the requesting patient. In this way, the provider confirms the received provider confirmation request. The second part of a handshake is performed to ensure the medical treatment. The provider agrees to perform the medical treatment as requested and defined by the prescription to the providers location and the requested time period. After receiving the confirmation signal, the treatment machine may be unlocked. Further, the treatment machine may be prepared for the medical treatment (load the program and equip with required consumable according to the prescription).

In some advantageous embodiments, the method and checking validity comprises sending a physician confirmation request to a physician's device. Checking validity may comprise checking the received initiation request. In this way, the treatment machine expects the requested treatment to be confirmed by a physician from a medical point of view. For example, the physician can confirm, whether the requested treatment is appropriate for the patient, respectively, the prescription has been issued for this patient. This increases security against abuse and incorrect and/or wrong treatment of the patients.

In some advantageous embodiments, the method and checking validity comprises receiving a confirmation signal of the physician's device, indicating that a treatment provider is confirmed for the requesting patient. In this way, the physician confirms the suggested and selected provider and/or treatment machine for performing the medical treatment.

In some advantageous embodiments, the self-treatment procedure comprises receiving or measuring patient related vital parameters from a set of sensors for checking eligibility of the patient for the requested medical treatment and in case of non-compliance with a set of pre-configured reference intervals issuing a failure message and initiating a vital data non-compliance procedure. In the context of the present disclosure, non-compliance is defined as the lack of cooperation or collaboration of the patient during the medical treatment, e.g., refusal of a therapeutic measure or failure to follow rules of conduct. The reasons for non-compliance are diverse and can be intentional or non-intentional by the patient. Possible factors may include forgetfulness (especially in older patients), lack of suffering (e.g., in hypertension), lack of disease insight, non/misunderstanding of the disease, insufficient or missing explanation of the measures by the physician, unpleasant side effects of the treatment, anxiety about side effects, stress, treatment costs, and/or religious reasons. The pre-configured reference intervals may be based on empirical values for the respective treatment and/or may have been developed through clinical studies. They describe in which way and frequency the treatment has to be carried out in order to achieve the best treatment result. A failure message is a message in form of an electronic data signal comprising data about the treatment (e.g., patient, the treatment machine, performed medical treatment, treatment provider, location and date of the treatment) that a physician or a third party responsible for the medical treatment needs to analyze the issues and to make changes, so that the patient agrees with the medical treatment. A non-compliance procedure may comprise and performs actions to overcome the non-compliance. The patient eligibility test may be based on receiving or measuring patient related vital parameters from a set of sensors for checking eligibility of the patient for the requested medical treatment. The set of sensors may comprise various sensors for measuring vital parameter, e.g., weight, blood pressure, beat of the heart, body composition index, oxygen saturation, blood sugar and/or general impression of the patient, detecting and measuring the position and the environment around the position of the patient.

The body composition index may be output or provided by means of a body composition monitor (BCM). The BCM is a biomedical, electronic device that determines individual fluid status and body composition, with an assessment of clinically relevant parameters, comprising a quantification of fluid status (overhydration and total body water, V) and an assessment of body composition (lean tissue mass, adipose tissue mass) by using bioimpedance spectroscopy techniques. It may measure at 50 frequencies over a range from 5 to 1000 kHz to determine the electrical resistances of the total body water (TBW) and the extracellular water (ECW). For more technical details relating to the BCM, it is referred to WO 2002 036 004 A1.

The sensor can be located in the treatment machine, in wearables wearied by the patient, and/or mounted close to the position, where the medical treatment is performed. The check of the patient and receive of the vital parameters can be performed before starting the medical treatment. In this way, it can be ensured that the patient is in a condition that a medical treatment can be performed and/or the patient can treat him/herself. Further, the pre-check may reduce the risk that the patient loses consciousness and is not able to monitor and/or control the medical treatment.

In some advantageous embodiments, measured patient related vital parameters are compared with received patient related vital parameters for the purpose of verification and in case of deviations above a pre-configured threshold, corrections measures are initiated automatically. The received patient related vital parameter may be vital parameters measured by the physician and provided to the treatment machine for verification. Further, the received patient related vital parameter may be vital parameter measured with a preceding measurement. The verification has the advantage that changes in the vital parameters allow a conclusion to be drawn on the state of health in general, a change in the state of health, as well as a conclusion on the effect (positive, negative) of the treatment. The deviations that are permissible within the scope of the offset can be determined by the attending physician. If the measured vital parameter deviates from the pre-configured threshold, the measurement can be re-iterated and/or a message can be sent to an external instance and/or the pre-configured threshold can be adapted. In case of changed conditions for the medical treatment, it can be necessary to adapt also the threshold to perform the medical treatment. Further, as a result if the deviation is above or below a pre-configured threshold, the medical treatment may be refused, adapted, or aborted. The patient is asked to contact his doctor or to go to a hospital in an emergency situation. In case the medical treatment is to be adapted, other settings at and for the medical treatment machine may be applied (e.g., amending the ultrafiltration rate in combination with an amended time duration for the treatment).

In some advantageous embodiments, a consistency check is executed for the vital parameters which have been provided by manual input, wherein the consistency check is based on an additional measurement of the vital parameters by means of sensors, which may be provided in wearables or in the medical treatment machine or at a treatment provider. In this way, incorrect patient inputs can be automatically verified and adjusted accordingly. This prevents medical treatment being carried out on the basis of incorrect vital parameters. Furthermore, intentionally wrongly entered values can be detected and corrected. Thus, the treatment is only carried out if the vital parameter of the patient for this treatment at the current time corresponds to the specifications. The input may also be monitored by using video cameras, etc.

In some advantageous embodiments, patient's vital parameters are sent to a metabolic model for checking consistency and for approving the prescription and/or for improving the metabolic model and/or the prescription, based on currently measured vital parameters. The provided and/or measured patient's vital parameters can be sent to a metabolic model for checking consistency and/or for approving the prescription and/or for improving the metabolic model and/or the prescription, based on currently measured vital parameters. A metabolic model may be a digital representation of a patient. The metabolic model enables a comprehensive data exchange. The metabolic model may also contain simulations, algorithms that describe, influence, or provide services about the properties and/or behavior of the patient. The data stored in the metabolic model may be linked to each other to a linking that is impossible to realize in the real world, which allows different analysis. The data received by the sensor can be provided by a standardized file-format, e.g., XML to the metabolic model. A parser reads the file with the sensor and provides the data to the metabolic model. The model may comprise different structural model types, e.g., a hierarchical model, relational model, an object-oriented model, or a network model.

A hierarchical model is a data model in which the data are organized into a tree-like structure. The data are stored as records which are connected to one another through links. A record is a collection of fields, with each field containing only one value. The type of a record defines which fields the record contains.

The relational model is an approach for managing data using a structure and language consistent with first-order predicate logic, where all data is represented in terms of tuples, grouped into relations.

Object-oriented model is based on objects. The data are organized in the objects. An object models an object or term and contains, e.g., associated attributes. Attributes describe an object in more detail. Data and methods (the functions for accessing the data) are stored together in the objects.

The network model does not require a strict hierarchy and can therefore also map m:n relationships, i.e., a data set can have several predecessors. Several data sets can also be at the top of the hierarchy. There are usually different search paths to get to a specific data set.

The metabolic model may be implemented a neural network and may be trained by using treatment outcome data as kT/V or an ultrafiltration volume as well and hence can be used to suggest a prescription in order to achieve the treatment goal. Thus, the metabolic model may be used to amend or optimize the prescriptions of medical treatments. In addition, data of the patient, e.g., gender, weight, age, vital parameters may be used for training. In this way, the model is trained for medical treatments relating to a specific patient. A trained model may be suggesting a medical treatment plan on the basis of provided patient parameters.

In an embodiment, the model is trained by sensor data measured while performing a medical treatment. If a patient does not tolerate the medical treatment, indicated by changes in the vital parameter, a manual changing of parameter at the treatment machine is recognized by sensors and used to retrain the metabolic model.

In an embodiment, the metabolic model may be used to setup treatment machines and/or test and verify medical treatments.

In some advantageous embodiments, a consistency check is executed for the vital parameters which have been provided by manual input of the patient, wherein the consistency check is based on additional measurement of the vital parameters by means of sensors, which may be provided in wearables or in the treatment machine or at the provider. In this way, the input of a user can be verified. A wrong or incorrect input of vital parameter can be replaced by the measured vital parameters. Therefore, a medical treatment is only performed with valid and admissible vital parameters.

In some advantageous embodiments, the self-treatment procedure comprises establishing a communication channel to a treatment provider's device and/or to a hub system for transmitting messages. In this way, a communication connection can be established between the provider's device and/or the hub system and the treatment machine for data exchange. Further, a message, e.g., a preparation message may be sent to the provider in advance. The preparation message may comprise, e.g., instructions how to perform a transportation of the patient to the location of the provider, the time schedule for the medical treatment, or the consumable required to equip the treatment machine for performing the requested medical treatment. Using a communication channel and a preparation message may increase the performance level of the provider as well as the treatment machine and at the same time reduces idle times of the treatment machines. Further, the treatment machine may provide information for planned maintenance measurements. A planned maintenance measurement is a maintenance that has to be performed periodically and is based on specifications of the manufacturer of the treatment machine.

In some advantageous embodiments, checking validity comprises receiving result data of an executed patient eligibility test for self-treatment. The patient eligibility test may be based on checking a set of predefined patient certificates. Different certificates can be raised for different capabilities. The certification is processed by a certification function. A certification function is a procedure for certifying that a patient has or has acquired knowledge and capabilities with respect to required procedures or activities, related with a self-treatment procedure. The certification function may, e.g., relate to trainings or exercises which need to be passed by the patient. The certification function may, e.g., relate to a specific augmented or virtual reality training, to hygiene training, to a basic machine training, to a self-cannulation training, and/or to a self-treatment in general. Further certificates may relate to fault and alarm handling, to specific treatment machine certificates, to specific consumables certificates (proofing that the patient is certified in and capable of handling and operating with disposables) and/or to “end treatment” certificates.

The patient eligibility test may be further based on receiving or measuring patient related vital parameters from a set of sensors for checking eligibility of the patient for the requested medical treatment. The set of sensors may comprise various sensors for measuring vital parameter, e.g., weight, blood pressure, beat of the heart, body composition monitoring BCM (in this context it is referred to WO 2002036004 A1), oxygen saturation, blood sugar and/or general impression of the patient, detecting and measuring the position and the environment around the position of the patient. The check can be performed before starting the medical treatment. In this way, it can be ensured that the patient is in a condition to treat him/herself. Further, the pre-check may reduce the risk that the patient loses consciousness and is not able to monitor and/or control the medical treatment.

In some advantageous embodiments, the confirmation signal is received from a patient device, a hub system, a treatment provider's device and/or by a physician's device. In this way, the specific devices and systems can be used to confirm the initiation request and unlock the treatment machine. As the device can comprises handhelds, computers, laptops and the like, the party using the device is more flexible in place and time to provide the confirmation signal. Therefore, the treatment provider and/or the physician need not to be on site of the medical treatment.

In some advantageous embodiments, after unlocking and before operating the medical treatment machine, a preparation procedure is initiated for setting up the medical treatment machine and for loading a prescription. In this way, the treatment to be performed by the treatment machine is setup automatically by analyzing the prescription specifying the kind of medical treatment and its required settings.

In some advantageous embodiments, after unlocking and before or during operating the medical treatment machine, a treatment support function will be executed. The treatment support function may comprise a set of pre-configurable watchdog functions and may output operating instruction messages. Each patient has to be treated according to his/her medical treatment plan relating to the medical prescription. The treatment machine may perform different medical treatments. Each treatment may require a different technical setting (e.g., duration of treatment) and technical equipment (e.g., filter) of the machine. This setting can be derived by the machine setup support function from the request of the patient as well as of the prescription and thus efficiently provided. The technical setting of the treatment machine can be read and programmed automatically by the machine setup function. The hardware conversion (change or add of equipment) can be requested automatically and prepared or executed by a third party. For a self-treatment machine or if a third party is not available, the machine setup function may provide a digital manual, e.g., a manual in a PDF-format or a URL-link referencing to a specific website, to the patient, e.g., to the electronic device, explaining how to setup the treatment machine.

A watchdog function refers to a function for failure detection in the treatment machine. Further, the watchdog function may comprise a function to monitor specific physiological (e.g., movement, etc.) and/or vital data (e.g., consciousness, respiration, circulatory system) of the patient. If a possible malfunction is detected, the malfunction is either signaled to other components (e.g., displayed on the user interface of the treatment machine or the electronic device), sent to an emergency provider or physician. Further, if a malfunction in the treatment machine is detected, in case of a self-treatment, instructions are automatically provided on the user interface of the treatment machine or the electronic device to eliminate the failure. In case of a supervised treatment, the supervisor, e.g., a nurse or physician may be alarmed. In case of a critical malfunction, especially those which endanger the health of the patient, the medical treatment can be aborted and a physician is alarmed.

The watchdog function may receive data from specific sensors installed in the treatment machine and/or in the electronic device for monitoring specific functions of the treatment machine and/or the specific physiological and/or vital data of the patient. In an embodiment, a smart watch may comprise sensors for measuring the blood pressure, beat of the heart continuously. The sensors can also be included in an apparel that a patient has to wear while performing the medical treatment. Further, the sensors can be included in a pulse belt that a patient has to wear below his/her chest. Further, the treatment machine may comprise sensors to monitor the stream of the blood, the blood pressure in the treatment machine, the permeability of the filters, etc. Further, the treatment machine may comprise sensors to evaluate specific values for the blood, e.g., amount of water, blood sugar, values for secondary hemostasis, viscosity, blood volume, etc. If a malfunction of the treatment machine is detected by the sensors, the patient and/or emergency providers are alarmed depending on the degree of the malfunction.

In an embodiment, the watchdog comprises a specification of malfunctions that may occur during a treatment and how the patient and/or the emergency provider has to be alarmed. In a worst-case scenario, for instance, if a patient falls in sleep or becomes unconscious, and the monitoring by himself/herself is not assured, the treatment may be aborted by setting the treatment machine into a safe state and/or an emergency provider is alarmed. In a further embodiment, the sensor may comprise cameras, which are installed in the location, where the treatment machine is positioned and a medical treatment is performed. The cameras can be used to supervise the patient, e.g., in a self-treatment scenario to alarm an emergency provider when a malfunction occurs and the patient is not able to eliminate the malfunction or if he/she becomes unconscious. Further, the cameras can be used to supervise the location for performing the treatment, whether the medical treatments performed and the location comply with specific regulations (e.g., hygienical, maintenance, etc.).

In some advantageous embodiments, the treatment support function comprises a rating function for rating the machine, the treatment provider and/or for rating the medical treatment. In the present context, a rating is an ordinally scaled classification. The classification can be performed by a rating agency. Further, the classification can be performed by a patient, who has been treated. The rating can be displayed on a scale. The rating can be described with numbers or letters, wherein the highest rating starts with the number “1” or the letter “A” and lower ratings are described by higher numbers or further letter following the alphabetical order. Further combinations and/or orderings are possible.

A rating of the provider may comprise an examination and evaluation of the services provided and how the services, e.g., in which quality, quantity the services are provided. Further, the examination may comprise an examination of the medical treatment in the light of standardized services resulting in a ranking of the providers, e.g., registered providers, who are suggested to patients. In this way, a positive rating may signalize a provider, who fulfills and/or performs a service and/or a medical treatment according to the standards. A negative rating may signalize deficits in meeting the requirements of the standards.

A rating of a treatment machine may comprise an examination and evaluation of the medical machine used for performing the medical treatment. The examination may comprise an examination and evaluation of the technical state from the treatment machine or treatments that can be performed by the treatment machine. The evaluation may result in a ranking of the medical treatment machines. In this way, a positive rating may signalize a treatment machine that can perform all required treatments, is maintained, and technically in good condition. A negative rating may signalize deficits in meeting requirement for operating the treatment machine.

A rating of a medical treatment may comprise an examination and evaluation of the medical treatment with respect to the patient and his/her health status and the required medical treatment. In this way, a positive rating may signalize a medical treatment that may change the health status of the patient in a positive way and is well-tolerated. A negative rating may signalize deficits in meeting requirements for performing the medical treatment to the patient.

In some advantageous embodiments, the ratings for a provider, a treatment machine, and a medical treatment can be stored in a database. The database can be used to provide automatically specific results for specific queries relating to a provider, a treatment machine, and/or a medical treatment. The database can be stored on a server or a distributed server system that is connected to the shared medical treatment system by a communication connection.

In some advantageous embodiments, the medical treatment machine is hosted and administered by a treatment provider within a local network and may be part of a set of treatment machines of the same provider. In this way, a treatment provider may administrate his/her treatment machines independent of the location.

According to a second aspect, a computer program is provided. The computer program includes program elements which induce computer to carry out the steps of the method according to the first aspect and the aforementioned advantageous embodiments, when the program elements are loaded into a memory of the computer. The realization of the methods by a computer program has the advantage that already existing processing units, such as computer, handhelds, or server units can be easily adopted by updates in order to work as proposed by the disclosure.

According to a third aspect, a sharing enabler is provided. The sharing enabler, which is implemented on a medical treatment machine and which is configured to execute the methods.

### BRIEF DESCRIPTION OF THE DRAWING

The properties, features and advantages described above, as well as the manner they are achieved, become clearer and more understandable in the light of the following description and embodiments, which will be described in more detail in the context of the drawings. This following description does not limit the invention on the contained embodiments. Same components or parts can be labeled with the same reference signs in different figures. In general, the figures are not for scale. It shall be understood that an embodiment can also be any combination of the above embodiments.

In the following possible embodiments are described in more detail with reference to the enclosed figures.

The scope of the present invention is given by the claims and is not restricted by features discussed in the description or shown in the figures. Generally, any reference signs in the patent claims should not be construed as limiting the scope.

FIG. 1 is a schematic representation of a treatment machine connected to a shared medical treatment system;

FIG. 2 is a schematic representation of a treatment machine connected to a shared medical treatment system;

FIG. 3 is a flowchart of a treatment sharing method;

FIG. 4 shows a perspective view of a fluid conditioning system that can operate with a medical treatment machine, e.g., with a dialysis machine, to carry out a fluid conditioning cycle that includes a medical treatment;

FIG. 5 is a front perspective view of a hemodialysis system;

FIG. 6 shows an example of a peritoneal dialysis system;

FIG. 7 is a block diagram of more abstracted generalized representation of a computing entity according to an embodiment of a patient device;

FIG. 8 shows a schematic flowchart representing the requesting of a treatment machine;

FIG. 9 shows a schematic flowchart representing a daily check of a patient;

FIG. 10 shows a schematic flowchart representing the preparation of a medical treatment; and

FIG. 11 shows a schematic flowchart representing the medical treatment.

### DETAILED DESCRIPTION OF THE DRAWINGS

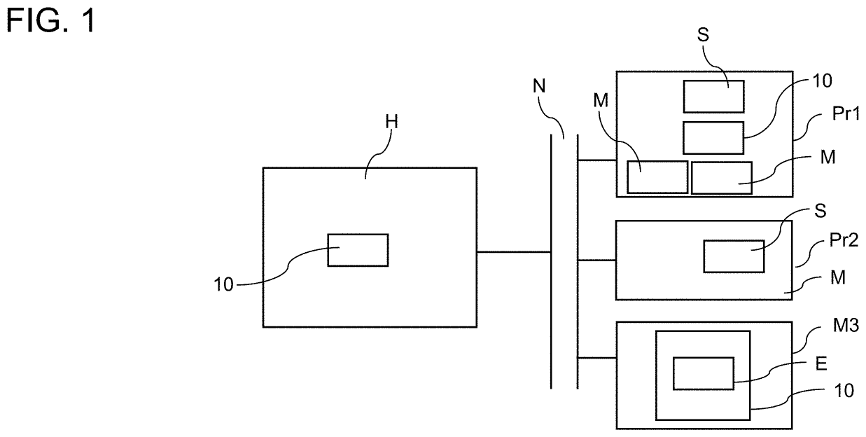

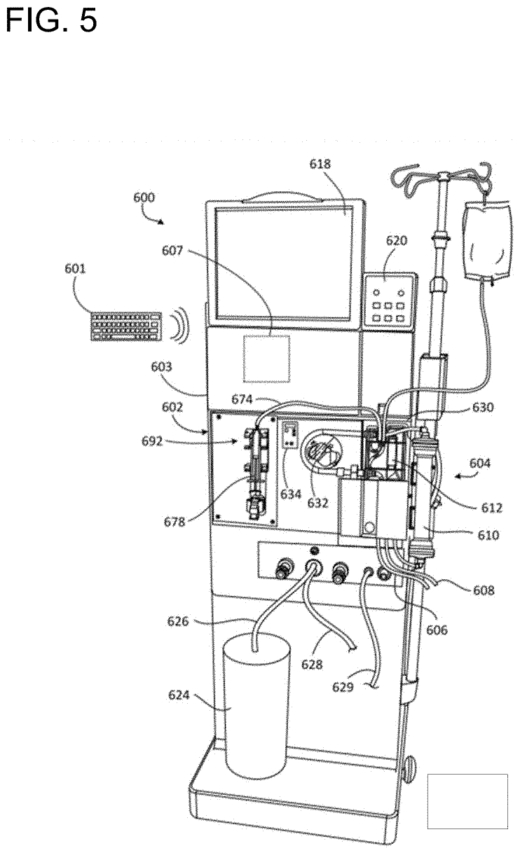

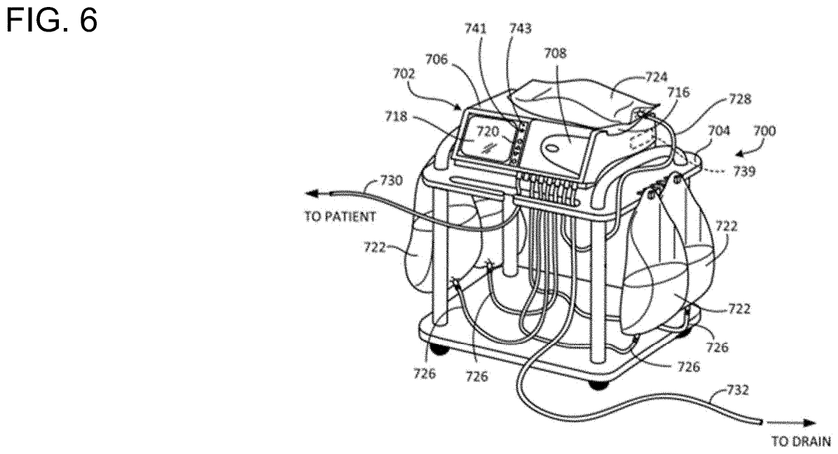

FIG. 1 shows an overview figure with a plurality of treatment providers Pr, respectively treatment machines, which may register in a shared medical treatment system. Each provider Pr and/or the treatment machines M may establish a communication connection to a network N for connecting to a central hub H of the shared medical treatment system. The central hub H is a computer entity and could be a server or a virtual computer, or a computer instance processed on a server or server system. The central hub H may comprise an interface for providing a communication connection to the network N. The central hub H may comprise a matching module MM that is implemented in a computing entity 10 (electronic module) for matching a patient Pa to a matching treatment machine M. A plurality of providers Pr and/or treatment machines may register to participate in the shared medical treatment system. A provider Pr may comprise at least one treatment machine M, e.g., a dialysis machine M, explained in more detail with respect to FIG. 5 and FIG. 6. A provider Pr or may be a hospital or a dialysis department or a private person that wants to offer the treatment machine M to other patients Pa for self-treatment is shown. A provider Pr may therefore provide at least one treatment machine M for medical treatment services. As presented in FIG. 1, provider Pr1 provides two machines M. Further, the provider Pr1 may provide sensors S, which are configured to detect environment data of the treatment machine M while performing the medical treatment, before, and/or after the medical treatment, for detecting, e.g., whether other persons or animals entered the treatment location. In case a person or animal entered the treatment location, a further disinfection may be initiated. Further, the provider Pr1 may provide or may linked with a computing entity 10 for processing a matching module MM, or solely a provider's part thereof (in the embodiment, where the matching module MM is implemented in a distributed manner). In this case, the other, e.g., a patient's part may be implemented and processed in the computing entity 10 of the central hub H or in the computing entity 10 of the patient device PaD.

In another preferred alternative embodiment, another setting of the provider Pr and the treatment machine M is applied, which is shown by way of example in FIG. 1 with provider Pr2. In this setting, the machine M is staffed with electronic modules, e.g., with local sensors S. The sensors S may be designed to detect the (correct) handling and usage of the treatment machine M while performing the medical treatment, and thus may indirectly provide information relating to the patient's cooperation and treatment behavior, e.g., how long a certain task lasts and whether or not the patient Pa is acting sufficiently precise by executing one step after the other without too long breaks and/or whether or not he or she fulfills all self-treatment requirements. In this case the sensors S are located locally at the machine M of provider Pr2. Nevertheless, in addition the provider Pr2 may also be equipped with sensors S for detecting other parameters, e.g., a loading and/or status of the different machines M of the provider Pr. Further, the sensors S of the machine can be configured to detect possible disturbance, and or providing data for a planned maintenance.

In another embodiment, another setting of the machine M is applied, which is shown by way of example in FIG. 1 with machine M3. This setting relates to the machine M3, which is usually equipped with a computer unit (CPU etc.). This computer unit may be configured to include the computing entity 10, which has implemented thereon the matching module MM or a provider's part thereof. In this setting, the machines M are staffed with more CPU power and are made more intelligent and do comprise the control software, e.g., the sharing enabler for participating in the sharing system by themselves. Thus, the patient device PaD may directly interact with the corresponding (sharing enabler) the machine M3 of a provider Pr.

Generally, the computing entity 10 and/or the matching module MM may be distributed over several computer hardware or nodes in the communication network. Thus, there does exist different parts or portions of one functional entity. The functional entity may, e.g., be implemented in software as an application in short app, and thus, with a patient device part app and with a machine or provider's part app.

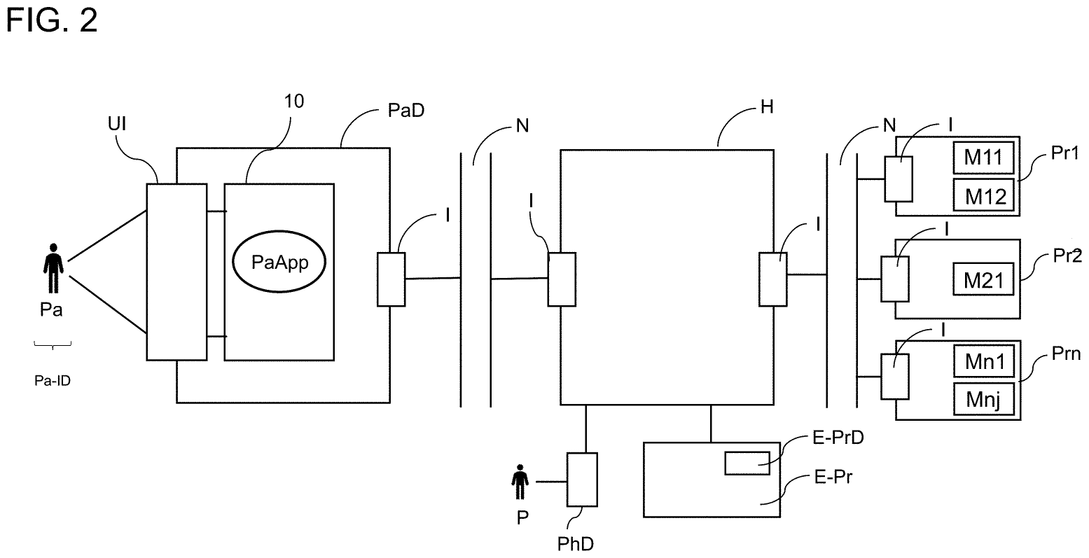

FIG. 2 shows an overview figure with a patient Pa using an electronic device, e.g., a patient device PaD. The patient device PaD may be an electronic wearable, e.g., a handheld or a smartwatch that can be used to register in a shared medical treatment system. The patient Pa is uniquely associated to a patient identifier Pa-ID. The patient identifiers serve to uniquely identify a particular patient Pa. The patient identifier Pa-ID may be an electronic code, e.g., in form of an electronic fingerprint, a data structure or a biometrical dataset. Generally, a patient Pa needs to register in the shared medical treatment system. Thus, not all patients Pa are registered patients. The patient identifier Pa-ID can be combined with an additional secret, e.g., a password comprising a random combination of numbers, letters, and/or characters. The password has to fulfill specific criteria relating to length and combination. The patient-identifier Pa-ID may be generated on or by means of the patient device Pa-D. Further, the patient identifier Pa-ID is associated to a password as a further part of the authorization credentials. The authorization credentials may be used to authenticate a patient Pa or the patient device Pad to other parties, e.g., computing entities 10, server etc. of the shared medical treatment system. In an embodiment, a two-factor authentication using a different communication channel for providing the second factor can be used. The patient device PaD preferably is a handheld or a smartwatch, but alternatively could be a computer, e.g., a laptop or workstation that includes respective interfaces, e.g., communication interfaces for connecting with the shared medical treatment system and its computing units.

The patient device Pad includes a user interface DI for communication to and from a patient Pa. The user interface can be configured as a touch screen interface providing input and output capabilities.

The touch screen can be used to authenticate and sign-in in a patient application PaApp processed by the electronic module 10, e.g. a processing unit. Further, the touch screen can be used to display the patient application PaApp. The patient application PaApp is designed for register or sign-in in the shared medical treatment system. Further the patient application PaApp provides capabilities for requesting a medical treatment, receiving a message with a suggestion for suggested treatment provider, and/or suggested treatment machine based on a matching algorithm. Further, the patient application PaApp may provide an eligibility check of the patient, a scoring and rating procedure for registered providers, treatment machines, and/or medical treatments. Further, the patient application PaApp may include a transportation support for getting to the providers location using an online map service provider, e.g., Google Earth, Google Maps. Further, the patient application PaApp may support a patient Pa for preparing the medical treatment or while performing the medical treatment with help instruction messages. Further, the patient application PaApp may alarm an emergency operator or physician in case of a medical emergency or malfunction of the treatment machine M. The patient application PaApp may implement a matching module performing a matching algorithm. Typically, the patient application PaApp is used by the patient him/herself. However, if he/she is not in the position to use the app, a trusted person (e.g., parents, or child, or family member) may use the app on behalf of the patient.

The patient device PaD may comprise further electronic modules 10, e.g., a memory for storing the operation system of the patient device PaD, the patient application PaApp, further applications, data measured by sensors of the patient device PaD or with the patient device, or connected sensors. Further, data concerning the entire medical treatment as well as data from sensors included in the treatment machine can be stored in the memory. The data can be stored in a database stored in the memory. Further, the data can be used to train a metabolic model that can be stored in the memory. Further, the patient device PaD may comprise further interface for connecting to the treatment machines M or to the provider Pr or to further computing units, e.g., a hub H, which can be implemented as a computing unit, e.g., a server. The interface I can be configured to implement a wireless communication connection, e.g., WIFI, Bluetooth, Infrared, etc. or a wired communication connection, e.g., serial communication connection.

The patient device PaD is connected to the hub H over a communication network N. The hub H can be implemented as central computer or server and/or virtual instance in server/cloud system. The hub H may comprise an interface I for connecting the hub to the communication network N. The interface I can be configured to establish a wireless communication connection, e.g., Wifi, or a wired communication connection, e.g., LAN. The hub H can be configured to process and store applications, e.g., a matching algorithm. In an embodiment, the matching algorithm may be implemented as an electronic module, e.g., a matching module for matching a patient Pa to a matching treatment machine M, which are depicted on the right-hand side of FIG. 2. In an embodiment, the matching module can be distributed and/or processed on different electronic modules in different hardware, e.g., a patient device PaD, a hub H, or a treatment machine M. In this way, the processing of the matching module MM can be split corresponding to the matching algorithm necessary for each party involved in the treatment sharing method. Further, the hub H may be configured to process the communication between the different parties.

A plurality of providers Pr may register to participate in the shared medical treatment system. Each provider Pr may comprise at least one treatment machine M, like a dialysis machine, explained in more detail below with respect to FIGS. 5 and 6. A provider Pr may be a hospital or a dialysis department or a private person, who wants to offer his or her dialysis machine M to other patients for self-treatment. A provider Pr may therefore provide at least one treatment machine M for treatment services. As shown in the example in FIG. 2, the provider Pr1 has two treatment machines M11, M12 and the second provider Pr2 has one treatment machine M21 and the n-th treatment machine provider Pm offers j treatment machines, Mn1, Mn2, Mnj. The treatment machine M may comprise an interface I that is configured to establish a wireless or wired communication connection to the network N for connecting to the hub H and/or the patient device PaD. The hub H may interact with a physician's device PhD. A physician Ph may use the physician's device to confirm the initiation request for unlocking the treatment machine M. The hub H may further interact to an emergency provider's device E-PrD. An emergency provider E-Pr may use the emergency provider's device E-PrD in case of emergency to support the patient Pa and/or the treatment provider Pr to prevent damage from the patient Pa and/or treatment machine M.

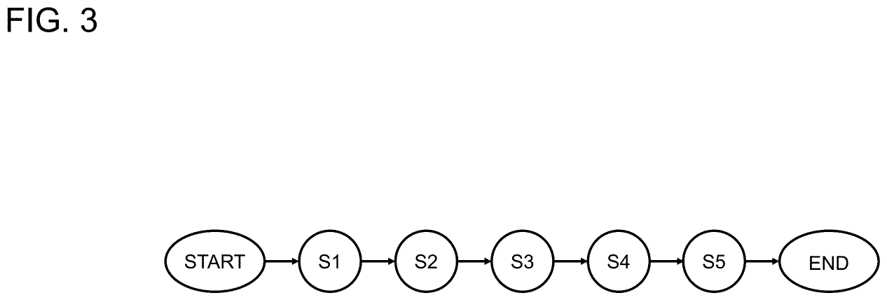

FIG. 3 is a flowchart of a treatment sharing method. The treatment sharing method, e.g., the sharing enabler E may be implemented on a computing entity 10 or of an electronic module of the computing entity 10. The computing entity 10 may be implemented in a registered treatment machine M, to be used in a shared medical treatment system. The sharing enabler E can be designed and programmed according to the operation system of the treatment machine M. After start of the sharing enabler, in step S1 a registration procedure for registering the medical treatment machine M in the shared medical treatment system is provided. In step S2 an initiation request for initiating a medical treatment for a patient Pa, being registered in the shared medical treatment system is received. In step S3, the received initiation request is checked, whether it is valid. The check is performed by receiving a confirmation signal. If the received initiation request is valid, the medical treatment machine M is unlocked, in step S4, in reply to the received confirmation signal for use for the patient Pa. Finally, in step S5, the medical treatment machine M is operated according to a treatment procedure in a sharing mode.

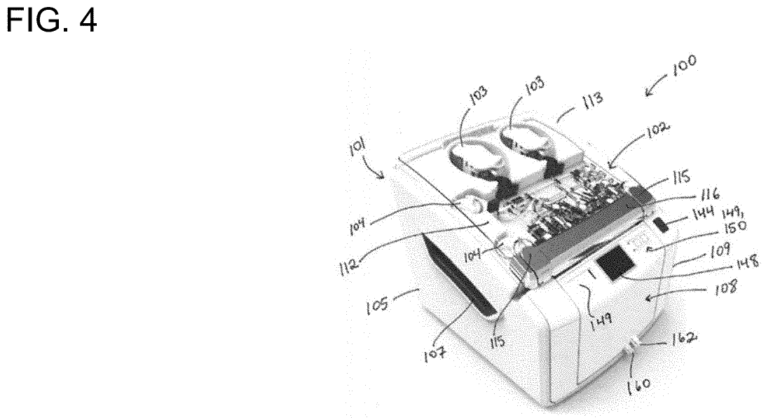

FIG. 4 illustrates a fluid conditioning system 100 that can be operated to prepare conditioned dialysate for use in a dialysis system. For example, the fluid conditioning system 100 can be fluidly communicated with the dialysis system to deliver “fresh” (e.g., cleaned, conditioned) dialysate to the dialysis system, collect “spent” (e.g., contaminated, unconditioned) dialysate from the dialysis system, and regenerate (e.g., cleanse) and condition the spent dialysate in a continuous fluid flow loop to recycle the spent dialysate. Example dialysis systems with which the fluid conditioning system 100 can be fluidly communicated include hemodialysis (HD) systems, peritoneal dialysis (PD) systems, hemofiltration (HF), hemodiafiltration (HDF) and other related systems.

The fluid conditioning system 100 includes a housing 101 that contains or supports components of the fluid conditioning system 100, a fluid cassette 102 that includes multiple fluid lines defining various fluid pathways, two relatively high capacity pumps 103 that can circulate fluid within the fluid lines of the fluid cassette 102, and two relatively low capacity pumps 104 that can deliver (e.g., infuse) conditioning agents into the fluid circulating within the fluid lines of the fluid cassette 102. The fluid conditioning system 100 has a compact footprint that facilitates lifting and transport of the fluid conditioning system 100. For example, the fluid conditioning system 100 typically has a length of about 30 cm to about 50 cm, a width of about 30 cm to about 50 cm, a height of about 30 cm to about 50 cm, and a weight of about 15 kg to about 20 kg. The housing 101 includes left and right side panels 105, 106, handles 107 positioned along the side panels 105, 106 for carrying the fluid conditioning system 100, a door assembly 108 that can be opened and closed to insert a heater bag, a front panel 109 to which the door assembly 108 is secured, rear and bottom panels 110, 111 that further enclose the interior components, an upper panel 112 that supports the fluid cassette 102 and the pumps 103, 104, and a cover 113 that protects the fluid cassette 102 and the pumps 103, 104. Example materials from which the exterior panels of the housing 101 may be made include plastics, such as acrylonitrile butadiene styrene (ABS) and polycarbonate blends, among others.

The cover 113 is typically made of ABS or polycarbonate and is transparent or translucent to allow visualization of the fluid cassette 102 and the pumps 103, 104. The cover 113 can be pivoted at a rear hinge 114 disposed along the upper panel 112 to open or close the cover 113. The upper panel 112 carries two latches 115 that can be closed upon a front edge 116 of the cover 113 to secure the cover 113 in a closed position. The latches 115 can also be pulled up and apart from the cover 113 to release the cover 113 from the closed position for accessing the fluid cassette 102 and the pumps 103, 104.

FIG. 5 shows a HD system 600 configured to wirelessly communicate with other medical devices. The HD system 600 includes a HD machine 602 connected to a disposable blood component set 604 that partially forms a blood circuit. The operator can manage and control treatment parameters of the HD system 600 using an external wireless keyboard 601. During HD treatment, an operator connects arterial and venous patient lines 606, 608 of the blood component set 604 to a patient. The blood component set 604 includes an air release device 612, which contains a self-sealing vent assembly that allows air but does not allow liquid to pass. As a result, if blood passing through the blood circuit during treatment contains air, the air release device 612 will vent the air to atmosphere. The blood component set 604 is secured to a module 630 attached to the front of the HD machine 602. The module 630 includes the blood pump 632 capable of circulating blood through the blood circuit. The module 630 also includes various other instruments capable of monitoring the blood flowing through the blood circuit. The module 630 includes a door that when closed, as shown in FIG. 5, cooperates with the front face of the module 630 to form a compartment sized and shaped to receive the blood component set 604. In the closed position, the door presses certain blood components of the blood component set 604 against corresponding instruments exposed on the front face of the module 630.

The operator uses a blood pump module 634 to operate the blood pump 632. The blood pump module 634 includes a display window, a start/stop key, an up key, a down key, a level adjust key, and an arterial pressure port. The display window displays the blood flow rate setting during blood pump operation. The start/stop key starts and stops the blood pump 632. The up and down keys increase and decrease the speed of the blood pump 632. The level adjust key raises a level of fluid in an arterial drip chamber. The HD machine 602 further includes a dialysate circuit formed by the dialyzer 610 various other dialysate components and dialysate lines connected to the HD machine 602. Many of these dialysate components and dialysate lines are inside the housing 603 of the HD machine 602 and are thus not visible in FIG. 5. During treatment, while the blood pump 632 circulates blood through the blood circuit, dialysate pumps (not shown) circulate dialysate through the dialysate circuit. A dialysate container 624 is connected to the HD machine 602 via a dialysate supply line 626. A drain line 628 and an ultrafiltration line 629 also extend from the HD machine 602. The dialysate supply line 626, the drain line 628, and the ultrafiltration line 629 are fluidly connected to the various dialysate components and dialysate lines inside the housing 603 of the HD machine 602 that form part of the dialysate circuit. During HD, the dialysate supply line 626 carries fresh dialysate from the dialysate container 624 to the portion of the dialysate circuit located inside the HD machine 602. As noted above, the fresh dialysate is circulated through various dialysate lines and dialysate components, including the dialyzer 610, that form the dialysate circuit. As will be described below, as the dialysate passes through the dialyzer 610, it collects toxins from the patient's blood. The resulting spent dialysate is carried from the dialysate circuit to a drain via the drain line 628. When ultrafiltration is performed during treatment, a combination of spent dialysate (described below) and excess fluid drawn from the patient is carried to the drain via the ultrafiltration line 629. The dialyzer 610 serves as a filter for the patient's blood. The dialysate passes through the dialyzer 610 along with the blood, as described above. A semi-permeable structure (e.g., a semi-permeable membrane and/or semi-permeable microtubes) within the dialyzer 610 separates blood and dialysate passing through the dialyzer 610. This arrangement allows the dialysate to collect toxins from the patient's blood. The filtered blood exiting the dialyzer 610 is returned to the patient. The dialysate exiting the dialyzer 610 includes toxins removed from the blood and is commonly referred to as “spent dialysate.” The spent dialysate is routed from the dialyzer 610 to a drain.

A drug pump 692 also extends from the front of the HD machine 602. The drug pump 692 is a syringe pump that includes a clamping mechanism configured to retain a syringe 678 of the blood component set 604. The drug pump 692 also includes a stepper motor configured to move the plunger of the syringe 678 along the axis of the syringe 678. A shaft of the stepper motor is secured to the plunger in a manner such that when the stepper motor is operated in a first direction, the shaft forces the plunger into the syringe, and when operated in a second direction, the shaft pulls the plunger out of the syringe 678. The drug pump 692 can thus be used to inject a liquid drug (e.g., heparin) from the syringe 678 into the blood circuit via a drug delivery line 674 during use, or to draw liquid from the blood circuit into the syringe 678 via the drug delivery line 674 during use.

The HD machine 602 includes a user interface with input devices such as a touch screen 618 and a control panel 620. The touch screen 618 and the control panel 620 allow the operator to input various different treatment parameters to the HD machine 602 and to otherwise control the HD machine 602. The touch screen 618 displays information to the operator of the HD system 600. The touch screen 618 can also indicate whether a peripheral or accessory device, such as the keyboard 601, is connected to the HD machine 602. The keyboard 601 is a wireless keyboard that connects to the HD machine 602 by communicating directly or indirectly with a communication system 607 in the HD machine 602. During treatment, the keyboard 601 and other peripheral devices can be used to control, monitor, and determine treatment parameters and variables.

FIG. 6 shows a PD system 700 that includes a PD machine (also referred to as a PD cycler) 702 seated on a cart 704. The PD machine 702 includes a housing 706, a door 708, and a cassette interface that contacts a disposable PD cassette when the cassette is disposed within a cassette compartment formed between the cassette interface and the closed door 708. A heater tray 716 is positioned on top of the housing 706. The heater tray 716 is sized and shaped to accommodate a bag of dialysate (e.g., a 5-liter bag of dialysate). The PD machine 702 also includes a user interface such as a touch screen display 718 and additional control buttons 720 that can be operated by a user (e.g., a caregiver or a patient) to allow, for example, set up, initiation, and/or termination of a PD treatment. Dialysate bags 722 are suspended from fingers on the sides of the cart 704, and a heater bag 724 is positioned in the heater tray 716. The dialysate bags 722 and the heater bag 724 are connected to the cassette via dialysate bag lines 726 and a heater bag line 728, respectively. The dialysate bag lines 726 can be used to pass dialysate from dialysate bags 722 to the cassette during use, and the heater bag line 728 can be used to pass dialysate back and forth between the cassette and the heater bag 724 during use. In addition, a patient line 730 and a drain line 732 are connected to the cassette. The patient line 730 can be connected to a patient's abdomen via a catheter and can be used to pass dialysate back and forth between the cassette and the patient's peritoneal cavity during use. The drain line 732 can be connected to a drain or drain receptacle and can be used to pass dialysate from the cassette to the drain or drain receptacle during use. The PD machine 702 also includes a control unit 739 (e.g., a processor), a speaker 741, and a microphone 743. The control unit 739 can receive signals from and transmit signals to the touch screen display 718, the control panel 720, the speaker 741, the microphone 743, and the various other components of the PD system 700. The PD machine 702 can receive audio input (e.g., spoken commands) through the microphone 743 and provide audio output (e.g., spoken alarms, alerts, and instructions) through the speaker 741. The control unit 739 can control the operating parameters of the PD machine 702, for example, based in part on the audio input and output. In some implementations, the control unit 739 may be an MPC823 PowerPC device manufactured by Motorola, Inc.

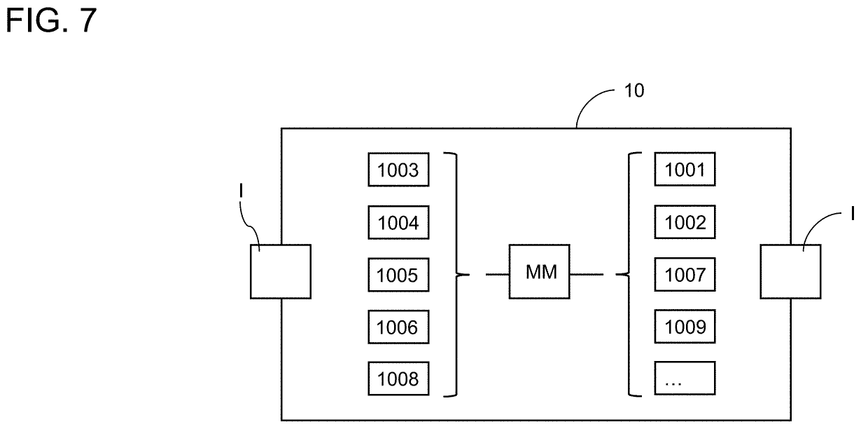

FIG. 7 is a block diagram of more abstracted generalized representation of a computing entity 10 according to an embodiment of an electronic device. In FIG. 7 the computing entity 10 is shown and explained with several electronic modules. The computing entity 10 may be a computer and/or an embedded device. The computing entity 10 may also be a processor or a processing unit. The computing entity 10 can be implemented in the patient device PaD, treatment machine M or in the provider's device. Usually, in a basic embodiment, the computing entity 10 at least comprises an interface I for establishing a communication connection and the matching module MM. The matching module MM may also be provided at least on part on another entity, e.g. on the provider Pr. Further, the computing entity 10 may be distributed over several hardware units or virtualized, respectively. In the following, some embodiments are described in common, e.g., comprising the modules and checkers 1001 to 1009. For a person skilled in the art, all or a part of these modules may be selected for incorporation and implementation on the computing entity 10.

The matching module MM is may be a part of a computing entity 10 or distributed to a plurality of computing entities 10. The matching module MM implements a matching algorithm that serves to flexibly associate or match a registered patient Pa and a registered treatment machine M at provider Pr. If a provider Pr only hosts one single machine M, then the matching algorithm matches a registered patient Pa to a provider Pr (which represents the machine M). The matching algorithm matches a first set of criteria relating to the patient Pa (e.g., reachability/nearness, availability of special services etc.) and/or with a second set of criteria relating to the machine M (e.g., if the machine requires patients Pa who know to use the machine M, who have, for instance, a disinfection certificate and a set up certificate).

The first set of criteria relating to the patient Pa and the second set of criteria relating to the machine M are stored in a database or in a distributed database system. The matching algorithm associates or matches the specific criteria provided by the patient Pa and the machine M. For instance, the patient Pa seeks for a medical treatment opportunity at a specific location in a specific time period. Using the treatment sharing method, the patient Pa may request a medical treatment by providing and/or selecting a location of a medical treatment and a specific time period for the medical treatment. This information is stored in a database. A provider Pr of a treatment machine M and/or the treatment machine M itself operates the medical treatment according to a certain schedule and has the knowledge when the treatment machine M is in use respectively is available. The available times periods can be stored in the database and are thus displayed and/or provided accordingly for incoming requests. With the user request, a query with the corresponding criteria for the medical treatment is started. If the requested time period corresponds to the time period, where a treatment machine M is available, the provider Pr of the treatment machine M and/or the treatment machine M is suggested to the patient Pa. This only represents a possible embodiment for searching an available treatment machine M according to a criterion. It is also possible to combine a plurality of criteria, which have to match before a provider Pr or a plurality of providers Pr are suggested to be selected by the patient Pa. In an embodiment, the patient Pa may specify, which number of total requested criteria has to be matched. In a further embodiment, the criteria may comprise a factor that describes a significance of the criteria for the patient Pa. The significance may comprise a ranking starting from a high significance to a lower significance.

The matching algorithm may be based on pre-defined criteria, which may be stored in a central database. The pre-defined criteria can be provided to the patient Pa for selecting treatment machines M according to his/her treatment requirements. In this way, standardized criteria are provided. In a further embodiment, the matching algorithm may consider reviews of the patients Pa (for the machine or provider), and/or of the provider Pr (for the patient). The reviews may be based on credit points or assessments of the respective users.

A verification module 1001 is specifically designed for verifying the matching result of the matching module MM. Generally, a verification is implemented such that another entity and/or another party verifies the matching result, which has been generated automatically by means of the matching algorithm. For instance, it can be defined that a patient Pa needs to input a verification signal on his patient device PaD. It may also be defined that a physician needs to input a verification signal on his physician's device PhD. Further, the provider Pr or the operator thereof may need to input the verification signal. All or a part of the embodiments for the verification may be combined, e.g., that a provider Pr and a physician P or the patient Pa need to verify the matching result. The verification may be based on different criteria, e.g. on a scoring, on another revaluation, on actual conditions and parameters (traffic, availability of resources etc.).

A scoring module 1002 is adapted for scoring the matching results based on pre-configurable scoring criteria. In a preferred embodiment, the scoring module 1002 comprises different parts or portions which may be implemented on different entities, so that different types of users may give a feedback for the matching result which has been provided automatically by the matching algorithm. For example, first, a patient Pa may score and evaluate the matching result, e.g., after the medical treatment at the treatment machine M has been finished. So, he or she may provide an input how good the selected machine M indeed has fulfilled his personal requirements. Second, a provider Pr may score and evaluate the matching result, e.g., after the medical treatment at the device or machine M has been finished with that patient Pa. The provider Pr may, e.g., score the punctuality and timeliness as well as, e.g., the reliability or other patient-related criteria. Third, a physician P, may score the matching result, after finishing the treatment and after being provided with the sensor measurements after the treatment. For instance, the measurements may indicate that possibly another type of machine M would be better to be used for that particular patient Pa. The scoring may be used by the matching algorithm as feedback for improving the matching effectiveness.

A certification module 1004 is designed for certifying a provider Pr and/or a patient Pa, participating in the shared medical treatment system. The certification module 1004 thus is directed to different users or entities of the shared medical treatment system, on the one hand the patient Pa and at least on the other hand the provider Pr. Different certification functions may be defined in a preparation phase for configuring the certification. E.g., an augmented and/or virtual reality training certificate, a hygiene training may be provided for providing and training the patient Pa with capabilities for using the treatment machine and for preparing him- or herself for the training. E.g., a self-cannulation test may be defined so that a self-cannulation certificate may be necessary and checked before the patient Pa is allowed to participate in the system. The self-cannulation certificate is an important security feature, as the skills for executing a self-cannulation need to be acquired. The process of cannulation refers to fluidly connecting the patient's bloodstream to the extracorporeal blood circulation. A blood vessel puncture, also referred to as cannulation, is a routinely required step in the medical treatment. Cannulation is performed in the medical practice by physicians or trained personnel. The quality of the vascular access created by the cannulation depends on a large number of parameters, which are characterized, e.g., by the individually and temporally variable abilities of the medical personnel and physical properties of the patients to be treated as well as by the diversity of the technical aids used in the puncture. Therefore, specific skills need to be acquired and proofed by way of a providing a self-cannulation certificate before being allowed to undergo a self-cannulation.

Certification functions may thus also be defined for the provider Pr, e.g., an eligibility checker certificate, a machine installation and setup certificate, a hygiene audit by a nurse certificate and others.

A consumable checker 1005 is configured for checking availability of required consumables for the registered patient Pa. E.g., a prescription may define that a patient Pa needs to use a specific type of consumables or disposables, like tube sets, filters, adapters etc. Thus, it has to be assured that this kind of type of consumables is available at the treatment machine M and, e.g., available at the scheduled treatment time. For instance, the consumable checker 1005 may also be configured to check storage of consumables at the local treatment machine M and to replenish stock of consumables, e.g., after each medical treatment. In an embodiment, the consumable checker 1005 may comprise a waste checker. The waste checker is configured to prevent fraud with respect to hazardous waste (e.g., of the filters used).

A maintenance checker 1006 may be configured for checking required maintenance and repair measures for all the treatment machines M within the provider Pr. Thus, this checker serves to provide an automated maintenance of the treatment machine M and this may in turn lead to an auto block in a hub calendar in case the maintenance checker 1006 has determined that the treatment machine M needs maintenance measures and need not to be used for further treatment. This data is to be collated and synchronized with treatment machine M booking and reservation data (of the scheduling system).

A prescription checker 1007 may be designed for checking whether prescription requirements may be fulfilled at the selected treatment machine M. This prescription checker 1007 is applied before the matching is done by executing the matching algorithm. In the physician's P prescription, special requirements may be defined for the treatment (e.g., a supervision is necessary, because the patient Pa is not fully capable for self-treatment and needs support by a nurse or medical assistant).

A transportation checker 1008 may be configured for checking whether transportation services are available for transporting the patient Pa to the selected treatment machine M or for transporting the treatment machine M to the patient's home, abode, or present residence. The transportation checker 1008 may be in message exchange with external services like public transport, private transport organizations and platforms (e.g., uber, my taxi etc.). The transportation checker 1008 preferably is in data exchange with the scheduling module 1003.

A selection module 1009 may be configured for selecting a preferred medical treatment machine M from a set of suggested medical treatment machines M. In an embodiment, the matching algorithm may generate a matching result with a set of medical treatment machines M for a particular patient Pa, who seeks an appropriate treatment machine M for self-treatment within the sharing system. With other words, the matching algorithm finds several potentially matching treatment machines M. Now, the selection module 1009 executes a selection function for finding the best option within the matching results for the specific patient Pa. The selection function comprises different selection criteria. The selection function may implement the different modules and checkers 1001 to 1009 as mentioned above and/or others.

In general, all the checkers and modules 1001 to 1009, mentioned above, may be in data exchange with each other.

The checkers and modules 1001 to 1009, mentioned above, are activated in different time phases:
The verification module 1001, the scoring module 1002, the prescription checker 1007 and/or the selection module 1009 are active after the matching result has been generated by the matching algorithm.
The scheduling module 1003, the certification module 1004, the consumables checker 1005, the maintenance checker 1006, the transportation checker 1008 are executed before the matching result is provided, e.g., during execution of the matching algorithm.

In a configuration phase the checker and modules 1001 to 1009, mentioned above, may be selected for usage in the shared medical treatment system. Further checkers may be defined, for instance a checker for detecting fraud with respect to the treatment machine M. Thus, special movement sensors (e.g. location-based sensors, GPS or the like) may be provided in the shared medical treatment system at the provider Pr and/or at the treatment machine M in order to detect any movement of the treatment machine M. If any unusual and incorrect movement is detected, a message will automatically be generated and transmitted to the provider Pr or to other external systems.

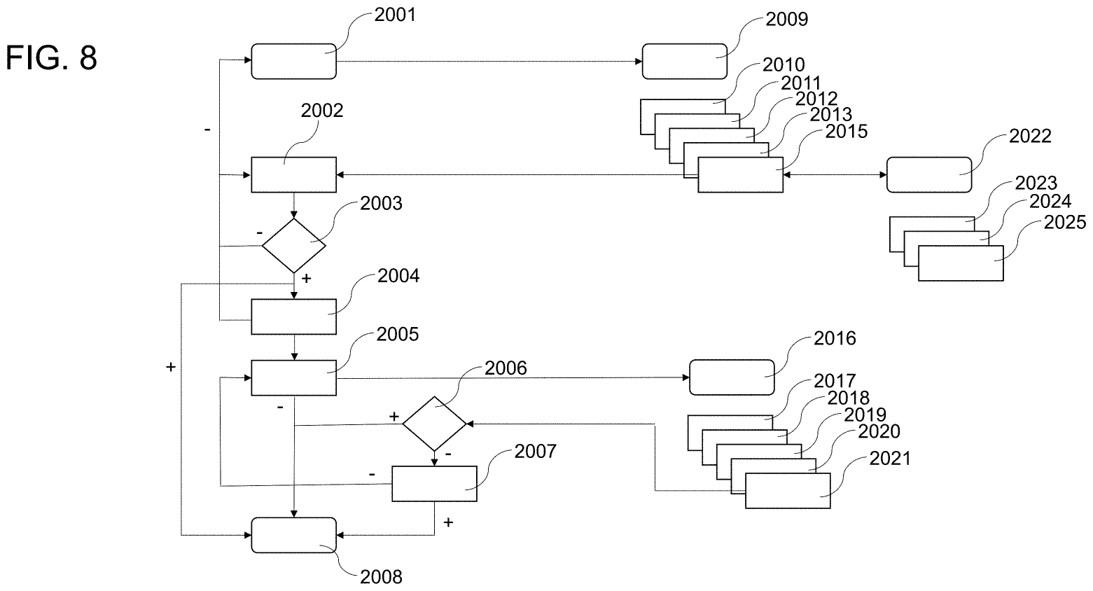

FIG. 8 shows a schematic flowchart representing the requesting of a treatment machine M. In step 2001 the treatment sharing method is started, e.g., the patient application PaApp is started that is used for requesting a medical treatment by a dialysis treatment machine M. In step 2009, a calendar can be displayed for the patient Pa at the patient device PaD. Further, the patent application PaApp may provide selection fields or buttons for further input and/or to specify the request and/or query, e.g., filters 2010. Further, in the patient application PaApp, available time slots 2011 are displayed. Additionally, the patient Pa can select the disposable 2012. The disposable selection can be administrated by a disposable module 2021. The disposable module 2021 may provide certified sets 2022. In an embodiment, the disposable module 2021 may prompt 2023 the patient Pa for bringing his/her own disposables, order the disposables with the provider Pr, or deliver 2024 it to specific address that can be entered by the patient Pa.

After selecting the disposable 2012, the patient Pa or an authorized person has to select the method of payment 2013, e.g., down payment, guarantee, etc. After finishing the payment selection 2013, a specific time slot for performing the medical treatment can be booked 2015. In step 2002 a specific time slot can be selected by a specific provider Pr. In step 2003, a confirmation of the selected provider and/or the treatment machine M is expected. If the selection of the patient Pa is cancelled by the provider Pr or treatment machine M (−) the requesting of a treatment machine M has to be re-started with step 2001 or another provider Pr is selected from a list, what avoids reinput of data. Alternatively, if the selection is not confirmed (−) by the provider Pr or treatment machine M, a reminder can be sent by the patient Pa to the provider Pr or treatment machine M in step 2004. If the reminder is not successful and the requested treatment is cancelled, the requesting of a treatment machine M has to be re-started with step 2001. If the request is confirmed by the provider Pr, the requested time slot with the requested provider or treatment machine M is booked in step 2008. Further, a long-term pre-booking of a medical treatment 2005 can be performed. If the patient Pa wants to repeat the medical treatment continuously, a calendar can be displayed 2016 in the patient application PaApp for selecting timeslots 2017, disposable 2018, and the method for payment 2019. A booking can be performed in step 2020. In step 2006, the provider Pr and/or treatment machine M has to confirm the request. If the requested is confirmed (+), the request time slot is booked for the patient Pa. If the request is not confirmed, a reminder can be sent 2007.

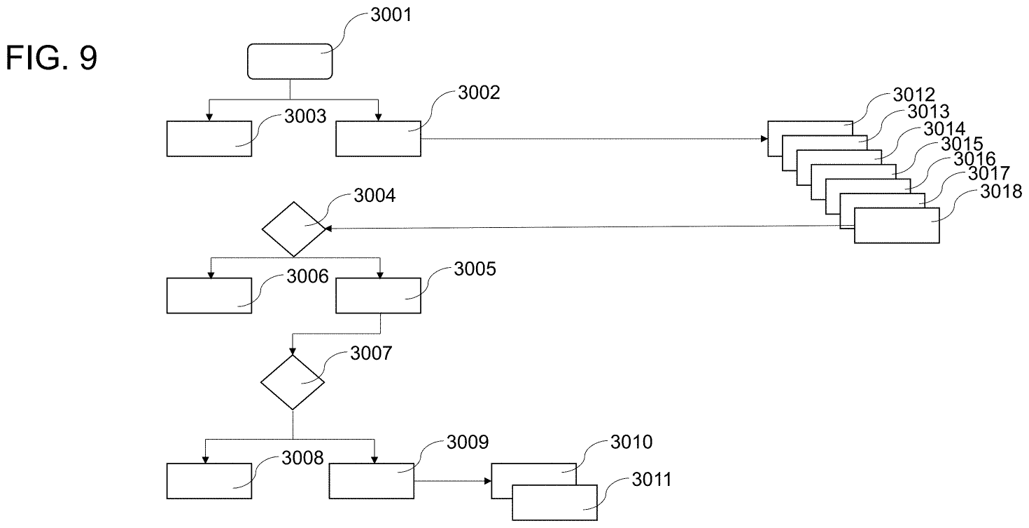

FIG. 9 shows a schematic flowchart representing a daily check of a patient Pa. The patient Pa has to perform a daily health check 3001 for evaluating his/her health status and to plan future medical treatments. The daily check may comprise a review from preceding medical treatments 3003. Further, in step 3002 the patient Pa can be asked to input specific data on a daily basis. The data can be input by the patient device PaD, and/or via a user interface of the treatment machine M, and/or via the provider device. The daily patient data (vital parameters) 3012 may comprise weight 3013, blood pressure 3014, fluid intake 3015, well-being 3016, take own picture 3017, the body composition index 3018. After the data is input in step 3004, an evaluation can be processed. The evaluation 3004 compares the patient data input with preceding patient data and/or preceding medical treatments (historical data). Based on the evaluation, a hospital visit can be suggested or the patient Pa may carry on with the daily routine. The evaluation may comprise a plausibility test. If the vital data of the patient Pa are out of range (−) a visit in the hospital is recommended 3006. The visit in hospital may comprise further steps, e.g., schedule date at hospital, send patient data to the physician P, and if a dialyses was booked for this day, the time slot at the provider Pr and/or the treatment machine M will be automatically cancelled. Advantageously, these steps can be performed by using the treatment machine M and the sharing enabler and/or the patient application PaApp installed in the patient device PaD. If the vital data of the patient Pa are in range (+), the patient Pa may continue with his/her daily routine 3005. If a medical treatment is booked (+) for that day 3007, the preparation for the medical treatment can be performed 3009. If no medical treatment is booked (−) for that day 3007, the day is off. The patient Pa can e.g., organize new consumables. If the medical treatment is booked 3010, the transportation 3011 has to be provided.

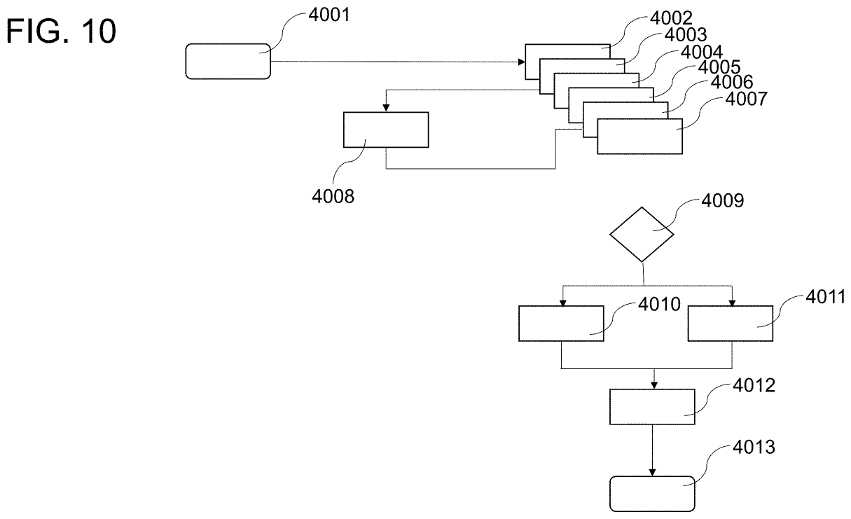

FIG. 10 shows a schematic flowchart representing the preparation of a medical treatment. In step 4001, the preparation for a medical treatment is started. The embodiment presented in FIG. 10 shows the preparation of self-treatment 4002. The self-preparation 4002 may comprise the measurement of the vital data 4003 (vital parameters). The vital data may comprise weight, blood pressure. The vital data are personal data, which have to be managed and processed in a secure manner. Further, the vital data may be used to train the metabolic model 4008 of the patient Pa. The metabolic model 4008 can be used for a consistency check with the daily input data. In step 4004, the treatment machine M can be setup. In step 4004, a medication can be supplied, if required. In step 4005, the metabolic model can be updated. In step 4007, the prescription can be uploaded and the time for performing the medical treatment can be adjusted.

Further, in step 4009 the metabolic model, comprising data of the prescription for a specific medical treatment can be adjusted according to the patient Pa, vital data, and/or preceding medical treatments. The adjusted prescription provided by the metabolic model has to be approved, e.g., by a physician P. If the prescription is approved, the patient Pa may use the adjusted prescription 4010 and if the prescription is not approved, the patient Pa may use the original prescription 4011. In step 4012, a self-cannulation can be performed by the patient Pa. After cannulation, the medical treatment can be started 4013.

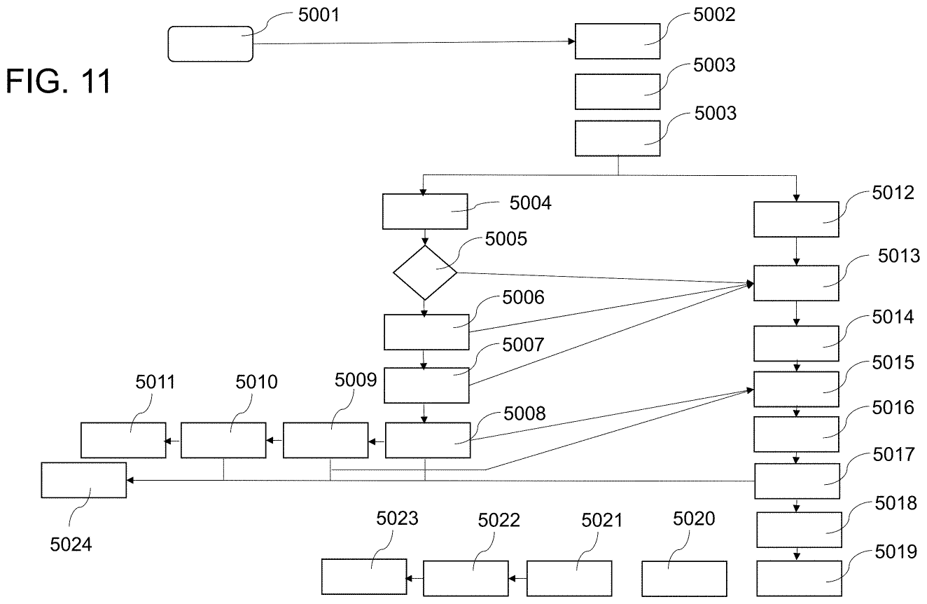

FIG. 11 shows a schematic flowchart representing a medical treatment. In step 5001, the medical treatment is started. After starting the medical treatment 5001, the medical treatment is performed for a specific time 5002. While performing the dialysis, the medical treatment is monitored by a watchdog 5003. The watchdog monitors continuously important parameters of the patient Pa and the treatment machine M. The watchdog can be configured to monitor and store specific parameter in a configurable interval. If a complication occurs during the medical treatment 5004, the patient Pa may be informed, respectively alarmed by the patient application PaApp or the treatment machine M. The patient application PaApp used with the patient device PaD or the treatment machine M may provide solutions to remedy the complication. If the patient Pa is able to solve the complications, the medical treatment continues in step 5013. If the patient Pa is not able to remedy the complication, the patient application PaApp or the treatment machine M may provide online help 5006. In this scenario, the patient application PaApp or the treatment machine M may connect the patient Pa to the online help, e.g., a provider Pr, a maintenance service, an experienced person, who can support. If the online help is available and the complication is remedied, the medical treatment continues in step 5013. If the patient Pa is not able to remedy the complication together with the online support, the patient Pa may call a technical support 5007. If the technical support may remedy the complication, the medical treatment continues in step 5013. If the technical support cannot remedy the complication, the medical treatment has to be aborted 5008. In step 5009, an alarm can be activated and a health check of the patient Pa has to be performed, because the required medical treatment has not been finished successfully. In step 5010, an emergency provider E-Pr has to be informed, because a dialysis which was not completed may lead into an emergency situation 5011. Further, if the medical treatment is aborted 5008 and an alarm is active, the physician P of the patient Pa is informed about the medical treatment. All data concerning the aborted medical treatment can be sent to the physician P.

If the medical treatment runs without any complication 5013, said medical treatment is finished in step 5014. In step 5015 a decannulation is performed. In step 5016, the treatment machine M is disarmed. The vital data of the patient Pa are controlled 5017 and can be sent to the physician P in step 5024. Further, the vital data and data measured while performing the medical treatment can be used to train the metabolic model. The treatment machine M has to be disinfected 5019 and the medical treatment has to be paid 5020. Further, in step 5021 the medical treatment, the provider Pr, and/or the treatment machine M can be rated. The rating can be used to provide a scoring for the selection process. Further, in step 5022 pictures of the treatment machine M and/or the location of the provider Pr can be taken to proof hygiene. In step 5023, the medical treatment is finished.

Wherever not already described explicitly, individual embodiments, or their individual aspects and features, described in relation to the drawings can be combined or exchanged with one another without limiting or widening the scope of the described invention, whenever such a combination or exchange is meaningful and in the sense of this invention. Advantages which are described with respect to a particular embodiment of present invention or with respect to a particular figure are, wherever applicable, also advantages of other embodiments of the present invention.

### REFERENCE NUMERALS

E Sharing enabler
E-Pr Emergency Provider
E-PrD Emergency Provider Device
H Hub
I Interface
In Input
Out Output
N Network
Pa Patient
PaD-M Patient Device Module
PaD Patient Device
PaApp Patient Application
P Physician
PhD Physician Device
Pr Provider
S1-S4 Method steps
UI User Interface
1001-1009 Modules and Checker
M Machine
S Sensor
10 electronic module
2001-2024 steps for requesting
3001-3018 steps for check
4001-4013 steps for preparation
5001-5024 steps for medical treatment

1-21. (canceled)

22. A method for executing a sharing enabler of a registered medical treatment machine, to be used in a shared medical treatment system, wherein the method comprises:
providing a registration procedure for registering the medical treatment machine in the shared medical treatment system;
receiving an initiation request for initiating a medical treatment for a patient being registered in the shared medical treatment system;
determining whether the received initiation request is valid by receiving a confirmation signal; and
in response to determining that the received initiation request is valid: (i) unlocking the medical treatment machine in reply to the received confirmation signal for use for the patient; and (ii) operating the medical treatment machine according to a treatment procedure in a sharing mode.

23. The method according to claim 22, wherein the medical treatment machine is hosted by a provider.

24. The method according to claim 22, wherein the medical treatment machine is hosted by a provider who is the patient.

25. The method according to claim 22, wherein the determining whether the received initiation request is valid further comprises checking availability of consumables necessary for executing the requested medical treatment and/or checking availability of necessary emergency providers for the requested medical treatment.

26. The method according to claim 22, wherein the operating the medical treatment machine is based on a prescription received from a physician's device via a network connection.

27. The method according to claim 22, wherein the registration procedure for registering the medical treatment machine comprises checking a set of certificates.

28. The method according to claim 27, wherein the method further comprises:
sending a provider confirmation request to a treatment provider; and
receiving a confirmation signal of the treatment provider indicating that the treatment provider confirms itself for the patient.

29. The method according to claim 27, wherein the method further comprises:
sending a physician confirmation request to a physician's device; and
receiving a confirmation signal of the physician's device, indicating that a treatment provider is confirmed for the patient.

30. The method according to claim 22, wherein the method further comprises establishing a network connection to a hub system, treatment provider's device, and/or to a physician's device.

31. The method according to claim 22, wherein the method further comprises:
receiving or measuring patient related vital parameters from a set of sensors for checking eligibility of the patient for the requested medical treatment; and
in case of non-compliance with a set of pre-configured reference intervals, issuing a failure message and initiating a vital data non-compliance procedure.

32. The method according to claim 31, wherein measured patient related vital parameters are compared with received patient related vital parameters for the purpose of verification and, in case of deviations above a pre-configured threshold, corrections measures are initiated.

33. The method according to claim 32, wherein a consistency check is executed for the vital parameters which have been provided by manual input, wherein the consistency check is based on an additional measurement of the vital parameters by means of sensors, which is be provided in wearables or in the medical treatment machine or at a treatment provider.

34. The method according to claim 22, wherein patient's vital parameters are sent to a metabolic model for checking consistency and for approving a prescription and/or for improving a metabolic model and/or a prescription, based on currently measured vital parameters.

35. The method according to claim 22, wherein the method further comprises establishing a communication channel to a treatment provider's device and/or to a hub system for transmitting messages.

36. The method according to claim 22, wherein the determining whether the received initiation request is valid comprises receiving result data of an executed patient eligibility test for self-treatment.

37. The method according to claim 22, wherein the confirmation signal is received from a patient device, a hub system, a treatment provider's device, and/or by a physician's device.

38. The method according to claim 22, wherein after unlocking and before operating the medical treatment machine, a preparation procedure is initiated for setting up the medical treatment machine and for loading a prescription.

39. The method according to claim 38, wherein after unlocking and before or during operating the medical treatment machine, a treatment support function will be executed, which comprises a set of pre-configurable watchdog functions and may comprise output of operating instruction messages.

40. The method according to claim 39, wherein the treatment support function comprises a rating function for rating the machine, the treatment provider, and/or for rating the medical treatment.

41. The method according to claim 22, wherein the medical treatment machine is hosted and administered by a treatment provider within a local network and may be part of a set of treatment machines of the same provider.

42. A computer program with program elements which induce computer to carry out the steps of the method according to claim 22, when the program elements are loaded into a memory of the computer.

43. A sharing enabler which is implemented on a medical treatment machine and which is configured to execute a method according to claim 22.
